(19) United States
(12) Patent Application Publication
(10) Pub. No.: US 2006/0031095 A1
(43) Pub. Date: Feb. 9, 2006

(54) Clinical workflow analysis and customer benchmarking

(76) Inventors: Axel Barth, Erlangen (DE); Dieter Boing, Muhlhausen (DE); Gregor Malischnig, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 10/913,224

(22) Filed: Aug. 6, 2004

(51) Int. Cl. G06Q 10/00

(52) U.S. Cl. 705/2; 705/7

(57) ABSTRACT

A method and apparatus is provided for analysis of the utilization of a technical medical device, such as a computer tomography, by a medical facility is provided. Recording of the use of the device is performed with time stamp information and a comparison of the use is made to other users of similar equipment or to similar users of different equipment. Optimization of use, such as through the purchase if different equipment and/or software or upgrades to the equipment and/or software is proposed.

### BACKGROUND OF THE INVENTION

1. Field of the Invention

The present invention relates generally to a method and apparatus for analysis of clinical work flow such as in a medical clinic and, in particular, to a method and apparatus for analyzing workflow of use of a technical medical device in a medical facility.

2. Description of the Related Art

Medical clinics addressing patient health concerns are required to purchase expensive equipment, handle often heavy patient loads, and operate within a budget. The expensive equipment should be fully utilized in order to maximize patient benefit, reduce overhead costs, and maximize return on investment. Current attempts to analyze utilization of the expensive technical equipment in the medical clinic includes preparation of user reports which are referred to as utilization management reports containing pure user statistical data such as operating hours, operating days, number of examinations, patient throughput, average examination duration, average time between two examinations, and scan seconds. Even if efforts are expended to expand this report to provide additional data from customers, these reports cannot provide the clinical workflow analysis and benchmarking.

### SUMMARY OF THE INVENTION

The present invention provides a method and an apparatus by which clinical workflow is analyzed and benchmarking of workflow is provided for clinical treatment of patients for example in a computer tomography apparatus or other technical medical scanning device. Data is collected with time stamp information for the workflow steps and are analyzed for possible improvement. A display of the time lines of the workflow events, either per patient process or in a combined (for example, averaged) view is provided in some embodiments. Utilizing the present invention with its expanded analysis and evaluation capabilities enables bottlenecks to be identified and recognized and improvements to be realized in clinical workflow. The present invention provides not only statistical evaluation but also provides a graphically prepared analysis of the clinical workflow. Workflow is displayed in a graphical display to operators and managers of medical clinics.

According to preferred embodiments of the invention, the computer tomography scanner or other technical medical device is operated and, during its operation, the workflow related events are recorded and stored in a data file with a time stamp. A targeted analysis of the data is performed to obtain retrospective information about the functions of the medical clinic. Evaluations of the workflow are displayed in a report in which the data is statistically evaluated and graphically prepared to graphically reduce the data for display to the clinic management relating to the clinic functioning and possible issues for improvement.

In a further, and potentially separate, aspect of the invention, the present method enables the quality of the workflow to be compared between medical facilities, in particular as it relates to use of the technical medical device. This is achieved by a benchmarking engine, utilizing as one of its core components an expanded workflow analysis. The survey data is combined into clusters of similar customer profiles depending upon the issue being addressed. Within each cluster, national as well as international benchmarking and ranking can take place. In other words, a best practices medical facility and a worst practices medical facility are identified along with the ranking of those between.

### BRIEF DESCRIPTION OF THE DRAWINGS

FIG. 1 is a functional block diagram showing the method and apparatus for workflow analysis and benchmarking according the principles of the present invention;

FIG. 2 is a representation of a graphical display of a workflow event browser of the present invention;

FIG. 3 is a graphical display of a standard workflow analysis;

FIG. 4 is a graphical display of a system workflow browser according to the present invention;

FIG. 5 is a graph of price recommendations for utilization management of magnetic resonance or computer tomographic utilization;

FIG. 6 is a graphical display of pricing recommendations for purchase of a magnetic resonance/computer tomographic apparatus utilization;

FIG. 7 is a graphical display of utilization of a magnetic resonance/computer tomographic apparatus;

FIG. 8 is a graphical representation of market analysis showing greatest need for utilization management for magnetic resonance/computer tomographic apparatus;

FIG. 9 is a table showing workflow parameters relevant to patient examination using a computer tomography apparatus in a medical clinic;

FIG. 10 is a table showing hardware utilization for a computer tomography apparatus in a medical clinic;

FIG. 11 is a table showing utilization information regarding a computer tomography apparatus in a medical clinic;

FIG. 12 is a table showing patient action and software background processes according to the present invention; and

FIG. 13 is a table of statistical parameters for use according to the present method.

### DETAILED DESCRIPTION OF THE PREFERRED EMBODIMENTS

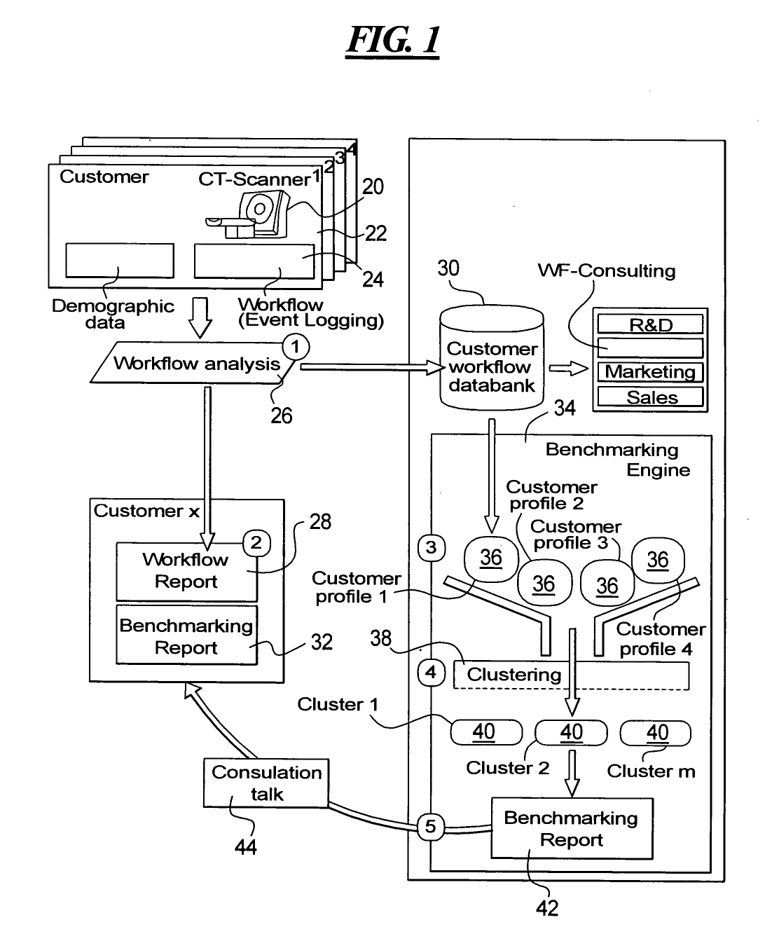

Referring to FIG. 1, an item of technical equipment in a medical facility, such as a computer tomography scanner or a magnetic resonance scanner where it presents a substantial investment for the medical facility, is used in the medical facility to diagnose and treat patients. The medical facility desires to fully utilize this costly equipment according to the patient load available at the medical facility. The illustration of FIG. 1, shows a computer tomography scanner 20 which is operated by a medical facility, here represented as a customer, in conjunction with an evaluation program 22 which starts at a defined point in time and searches through event logs according to specific parameters, thereby counting events, grouping results, setting results in relation to other parameters, and finally, storing the results in a data file. This is identified as workflow event logging 24. The resulting file is used as a basis for a workflow analysis 26 to generate a workflow report 28. The evaluation can be performed on a daily basis or at other arbitrarily defined time intervals. It is important that a results file is available and so the results are stored in a workflow database 30 relating to the computer tomography scanner. The results file must be retrievable over a defined time span to enable the analysis to be performed. The evaluation program should be remotely updatable to be able to react flexibly to new inquiries and evaluations.

In one embodiment, the data files are transferred as indicated by the arrows in the illustration of FIG. 1 via an existing network such as an office network, the internet, or other network or via a telephone or wireless connection to a central customer workflow database 30 to be available for expanded automatic workflow analysis over an entire installed base. Where such a network connection is not supported, it is possible that the data may be read out locally or even to locally implement a complete workflow analysis at the clinic site.

A workflow report 28 is provided by the analysis of the event logging for the computer tomography apparatus. The results file is evaluated for different complimentary types. A goal of the analysis is to provide to the clinic a comprehensive insight into the workflow at the clinic. Utilizing the present method, the possibility is provided to recognize optimization potentials and to adapt clinic functioning to these optimized potentials. For example, it may be desired to reduce the duration of the examination times to enable a higher patient through put or even to reduce the time that the computer tomography scanner is in operation. By analyzing the workflow report, it is possible to direct attention to the those function blocks which are longer than average and to target pauses in the workflow since these provide the possibility for the desired improvement.

The workflow report 28 may be comprised of a plurality of components. One such component is a short report. The short report is a first step which is created from the results files which are received from the clinic. A statistical preparation is provided in the form of tables from which the utilization or a load on the computer tomography scanner is visible by a brief overview.

Also shown in FIG. 1 is a benchmarking function. The benchmarking function is provided by accessing a clinic or customer, workflow data bank in which the profiles of all of the clinics, or customers, in question are stored. A mutual comparison is performed by drawing upon the data in the data bank to generate a benchmarking report 32. In particular, a benchmarking engine 34 draws the information from the workflow data bank 30 including customer profiles 36. The customer profiles 36 include demographic on the customer or clinic. Examples of such demographic data include the type of installation, such as the established radiologist, city clinics, university clinics, etc. The information also notes the hospital size, hospital category (such as private or state), the number of scanners, the product group (such as high-end product, mid-range product, or low-end product), the number of work stations, the archiving system, the specialization (such as whether the facility is children's clinic, cancer center, emergency room, etc.), the number of medical technical radiological assistance, etc. A clustering function is performed at 38. In particular, targeted analysis methods are applied when the workflow of a customer is to be compared to that of another customer, or clinic. This is possible when similar customer profiles which have either identical or similar demographic data and system configuration and similar examination types are combined into clusters. A certain bandwidth of perform and workflow data normally exists within these clusters. A ranking of customers according to specific criteria, for example, maximum patient through put in the shortest examination durations enables a comparison within the cluster. Once the clusters are defined, as shown at 40, the evaluation can begin. The larger a cluster (in other words the greater the number of members in the cluster) the more representative the evaluation. The clusters may be narrowed in order specify a search for a customer or clinic that achieves an optimal workflow (or best practice) with the same configuration as the other members of the cluster. The possibility to increase the efficiency via the workflow or via a system optimization can be provided from this analysis. Possible optimizations may be, for example, the shortening, restructuring, or parallelization (such as by the provision of a number of consoles) of the process steps.

The analysis of the cluster 40 results in a benchmarking report 42. The generation of a benchmarking report is provided by the access to the central customer workflow data bank 30. In the benchmarking report, the workflow of a customer, or clinic, is contrasted with the workflow of other customers or clinics within its cluster. A ranking of the clinics or customers within a cluster is performed so that it can be shown how the customer or clinic is positioned within the customer rank for the cluster. The preparation of the benchmarking report is followed by a consultation talk 44 with the customer, or clinic in which the benchmarking report 32 reaches the hands of the customer.

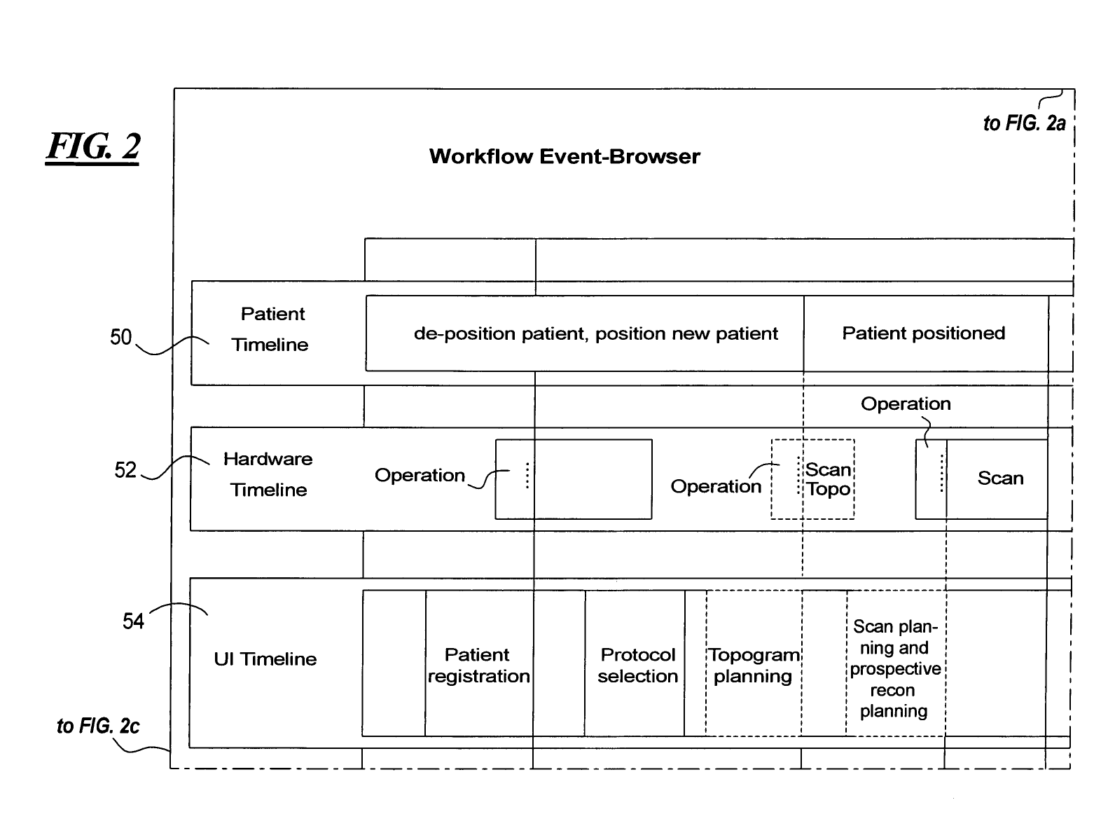

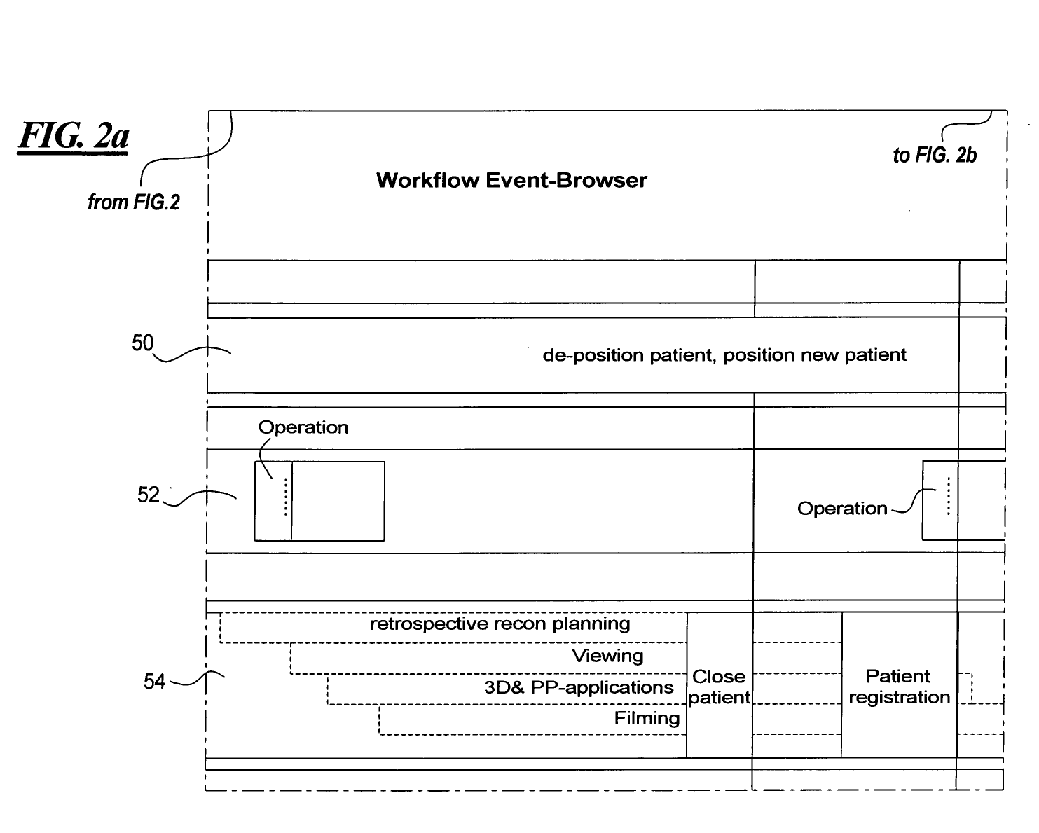

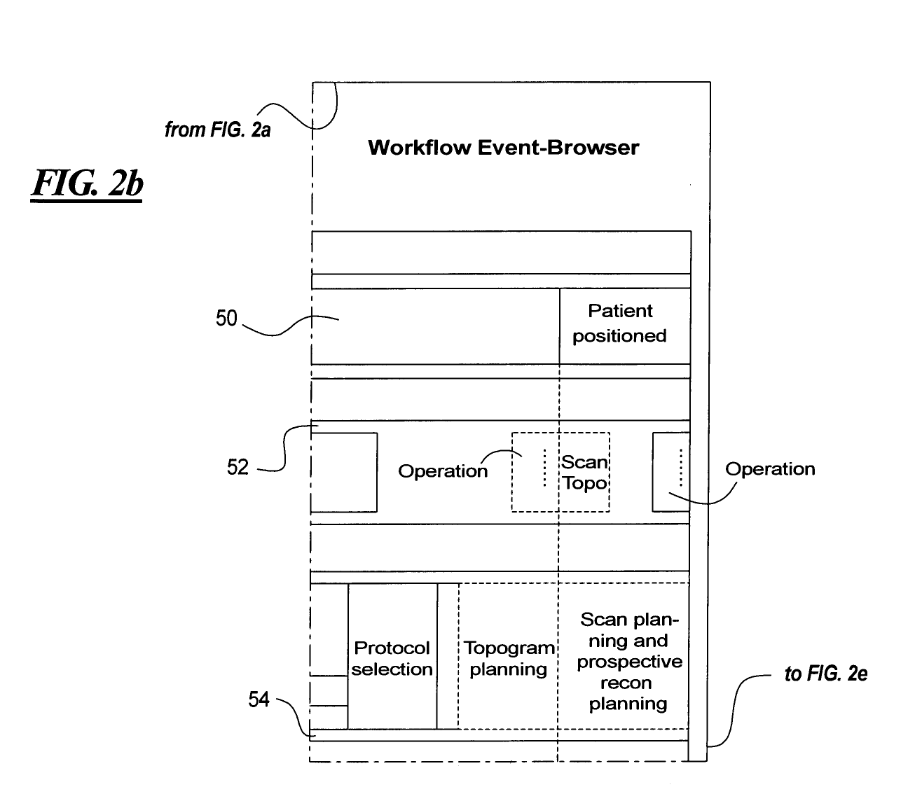

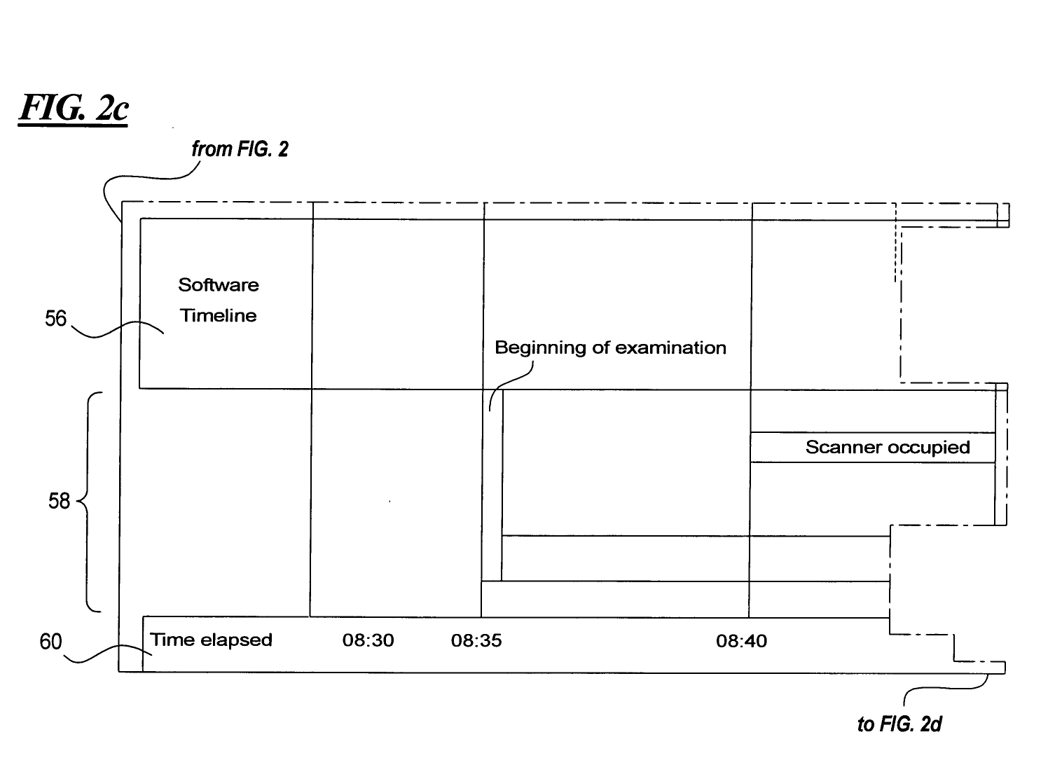

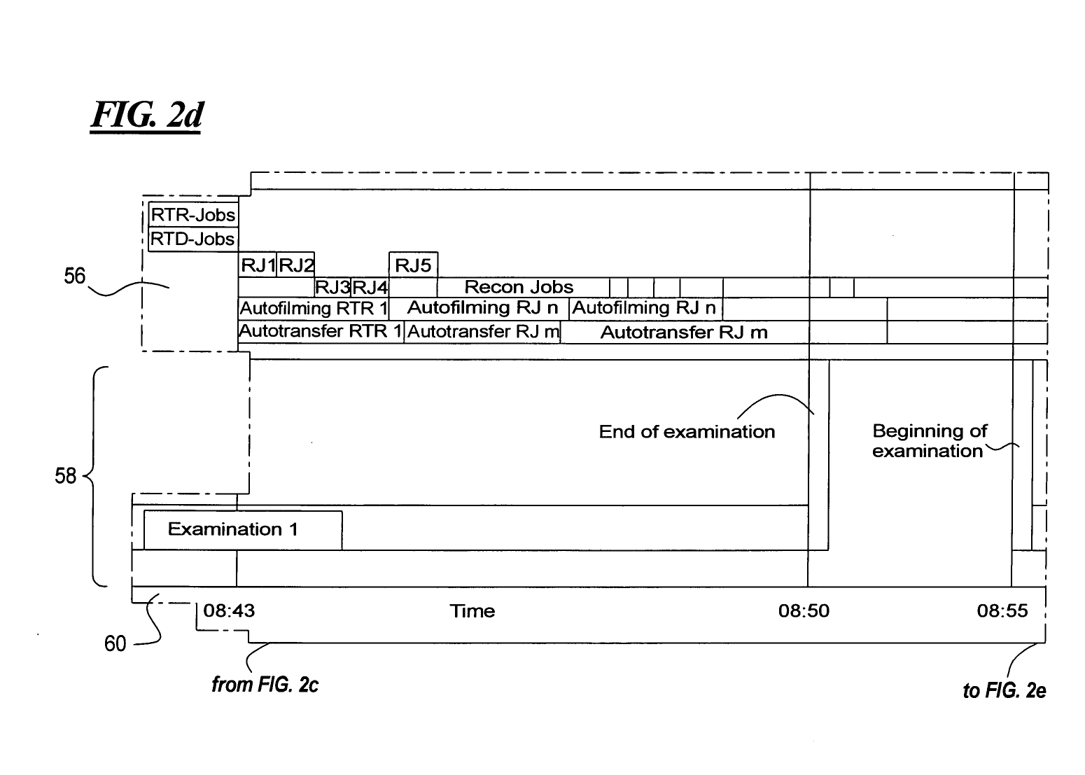

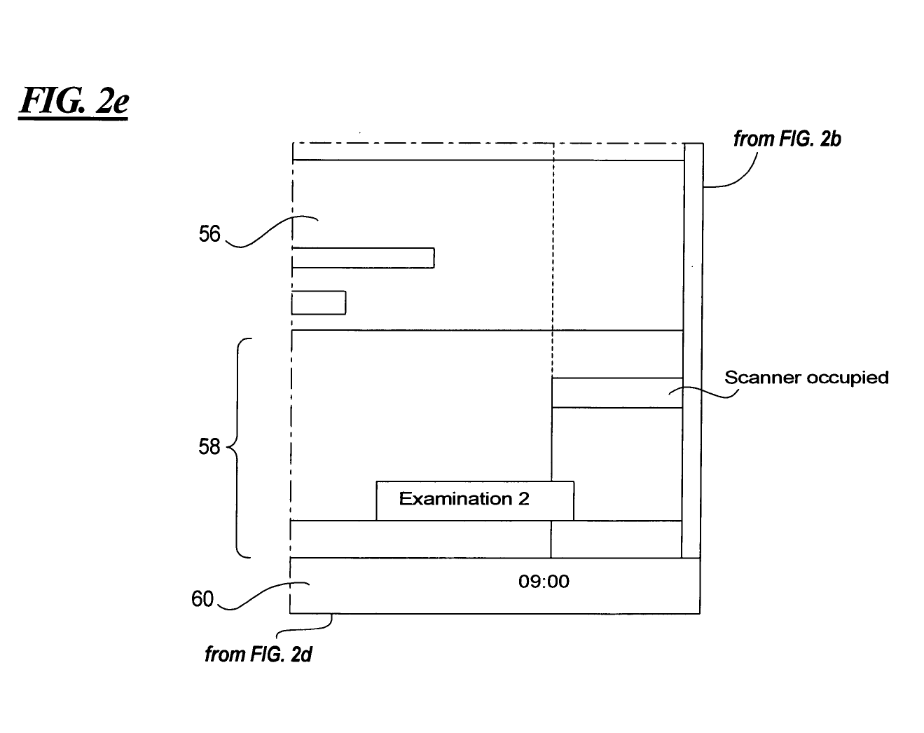

In FIGS. 2, 3 and 4, a display of a computer system is utilized to show a graphical representation of information relating to the invention. A standard personal computer, workstation computer or other type of computer display is possible to this aspect of the invention. It is also contemplated that display screens on equipment or other non-computer displays could be utilized in displaying this information.

Turning now to FIG. 2, the present invention utilizes a graphical display showing the workflow events performed during operation of the clinic. The graphical display as shown in FIG. 2 in which the customer may browse through the recorded events of the clinic operation. In the graphical illustration, the precise temporal course of examinations is displayed visually for specific selected time spans. In the example of FIG. 2, the time span of a day in February 2004, from 8:30 a.m. to 9:00 a.m. is shown. The examination steps are shown in parallel along four different time axes 50, 52, 54 and 56. The first axis 50 is the patient-based actions axis which refers to the positioning of the patient relative to the computer tomographic device. The second axis 52 is the hardware interactions timeline and refers to the operation of buttons in the computer tomograph for movement of the patient bed or radiation triggering. The third timeline 54 in FIG. 2 is the user interface (UI) or software interaction timeline. This refers to the manual input or graphical interaction by a user for planning the examination. The last timeline 56 illustrated is the software timeline which records the software functions, and specifically provides the duration of the software functions.

A summary timeline 58 is also provided which identifies patient processing activity that applies to all of the four timelines 50-56 lying thereabove. A bottom line 60 of the display plots the events against the actual time that they occurred.

The workflow event browser of FIG. 2 shows the extent of each step in the patient process, including clearly showing the pauses between the steps. The extent of each event is indicated in by a bar, although of course other types of displays of the events are also within the scope of the present invention. Each time bar is labeled to show the activity that has been carried out during that time. The viewer of the browser display is thereby able to easily see the duration of time that it has taken to prepare the patient for a procedure, or to position the patient, or to perform the scan on the patient. Pauses between patients at the scanning facility are also readily apparent, which may reveal problems that can be addressed to increase patient throughput, reduce patient waiting time, or better utilize the facility.

From the user interface interaction relating to the patient registration and to the examination transmission, the examinations can be distinguished from one another by reference to the patient using a unique patient examination identification. Additional information such as that on Mondays, only one medical technical assistant is on duty, or that a different workflow is performed on the early shift compared to the late shift, can also be recorded for display in the workflow event browser.

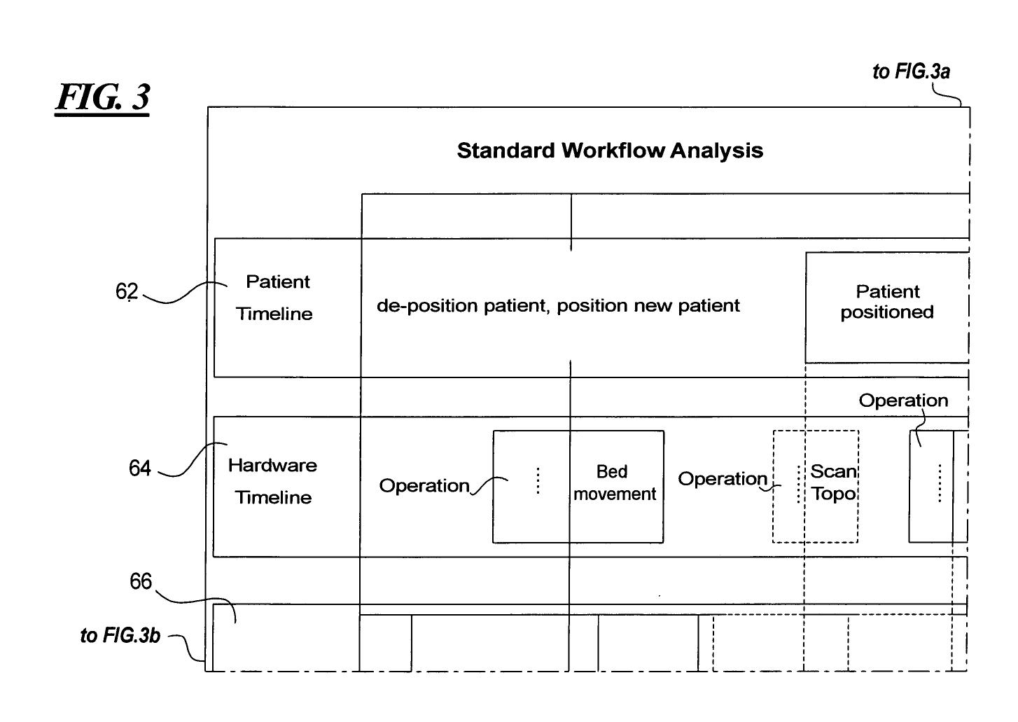

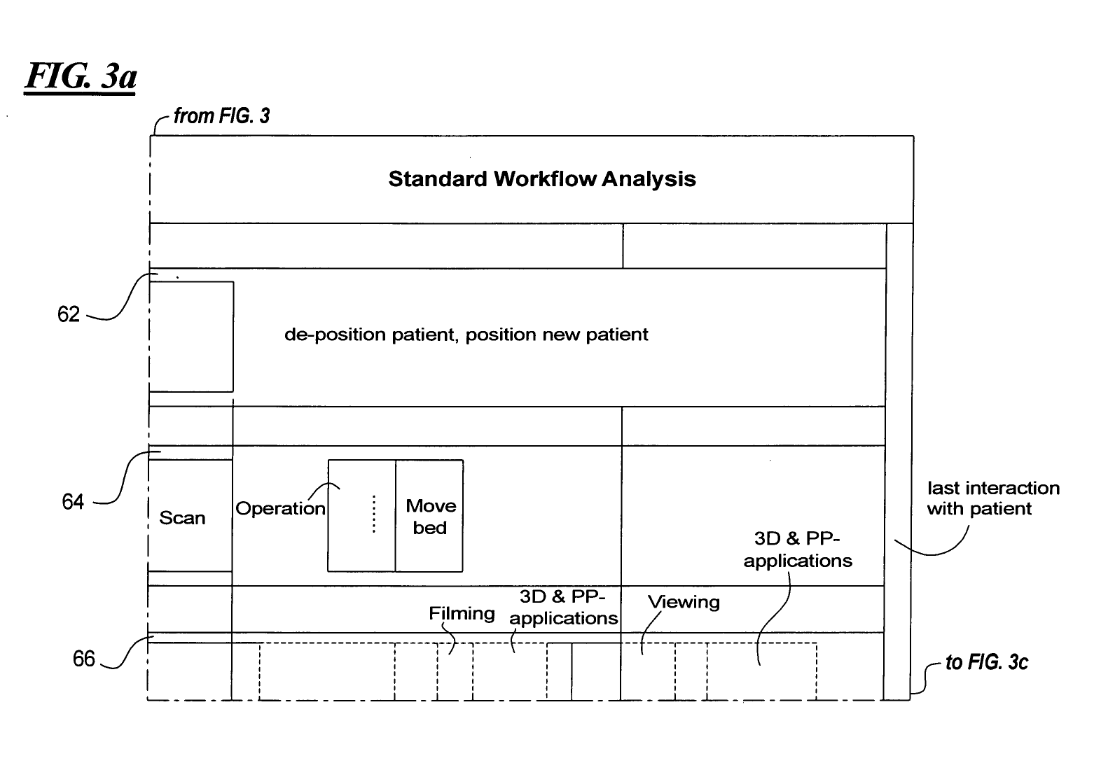

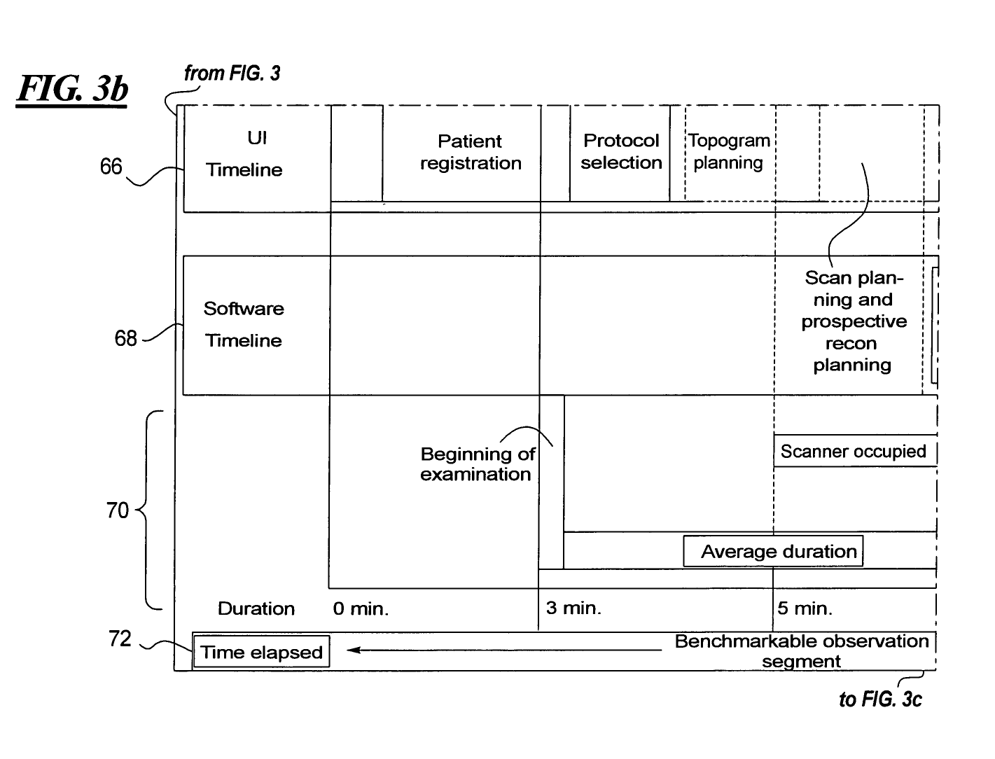

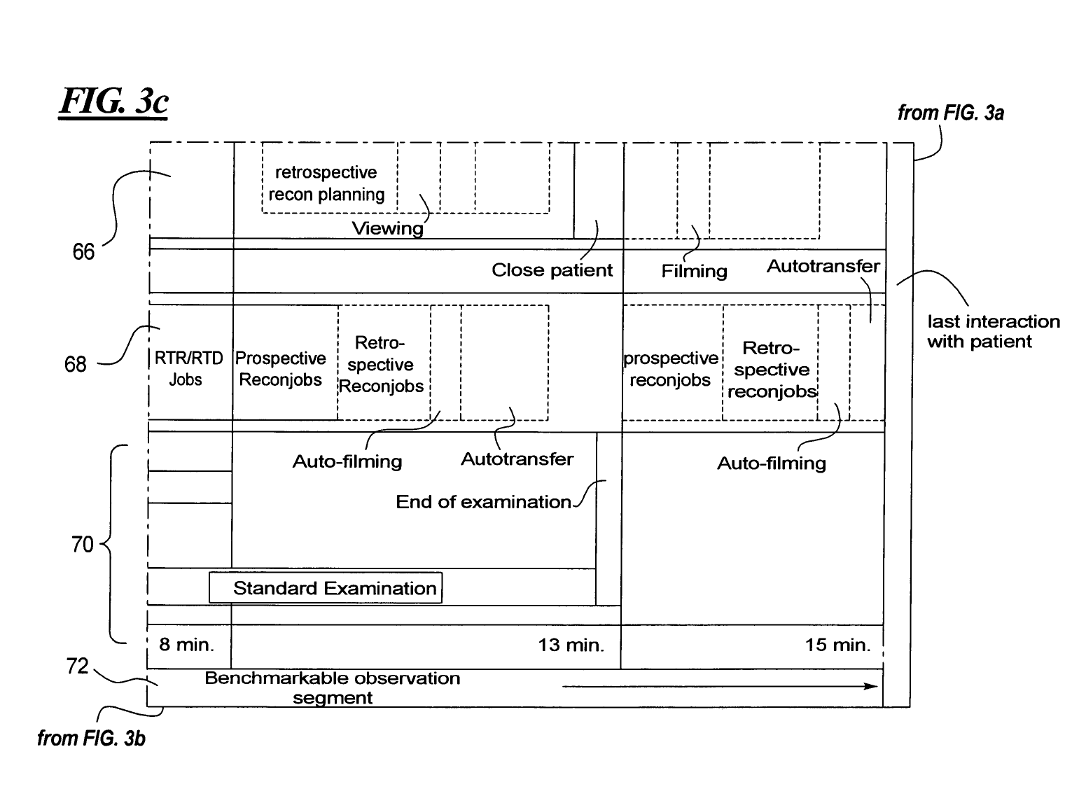

A standard, or combined, workflow analysis is performed according to another aspect of the invention. An example of a standard workflow browser according to the present invention is shown in FIG. 3. A precise statistical evaluation along with a graphical representation of the workflow is provided. This graphical representation mode merges the data of all of the examinations from an arbitrarily set observation, such as one day, one week, or one month, etc., and shows the combined data, for example as averaged function blocks over the time duration. The workflow events are shown in the same configuration and arrangement as in the workflow event browser of FIG. 2. In particular, four timelines are show including the patient timeline 62, hardware timeline 64, user interface timeline 66 and the software timeline 68. A summarizing timeline 70 and elapsed time timeline 72 are also shown. The standard or combined browser display shows the average times over multiple occurrences of each event, in contrast to the workflow event browser which shows each event that has been performed.

It is possible to select for review at a glance any desired type of examination, such as heart examination, standard thorax examinations, etc. The different types of examination may be visualized in the workflow analysis display using a standard format. In the illustrated workflow analysis of FIG. 3, the representation are in large part similar to the workflow event browser shown in FIG. 2. In the preferred embodiment, the representations are combined data provided along four timelines, although other numbers or arrangements of timelines are of course possible. By contrast, however, the absolute time relation of the events is not given due to the statistical averaging over a number of examinations.

Using the graphical illustrations of the workflow analysis shown in FIG. 3, it is possible to easily recognize longer than average function blocks or delays in the process and it is precisely this which permits improvements in the workflow process to be accomplished. For example, a clinic or customer may on average be occupied for a very long time with the positioning of a patient. This may be the result of a patient arriving for a computer tomographic examination without being prepared, such as without having an intravenous access. Another cause for delay could be that the scan parameter adaptations must be made immediately before conducting an examination do to insufficiently adapted examination protocols. If the examination steps are not adapted to one another, an unnecessarily long pause may be created and by recognizing such long pauses in the process, these delays can be recognized. It is a goal of an embodiment of the present method to recognize improvement potentials of a customer or clinic and to optimize the examination procedures so that individual function blocks and pause intervals are adapted to one another as quickly as possible and in an optimal manner.

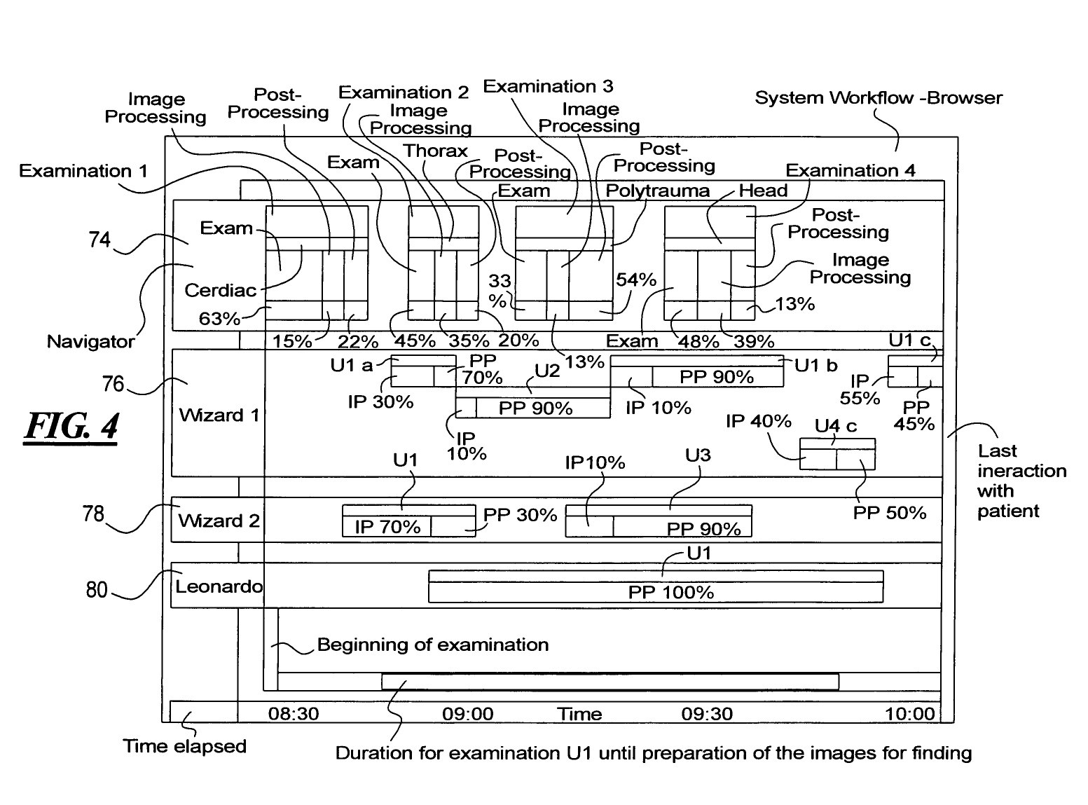

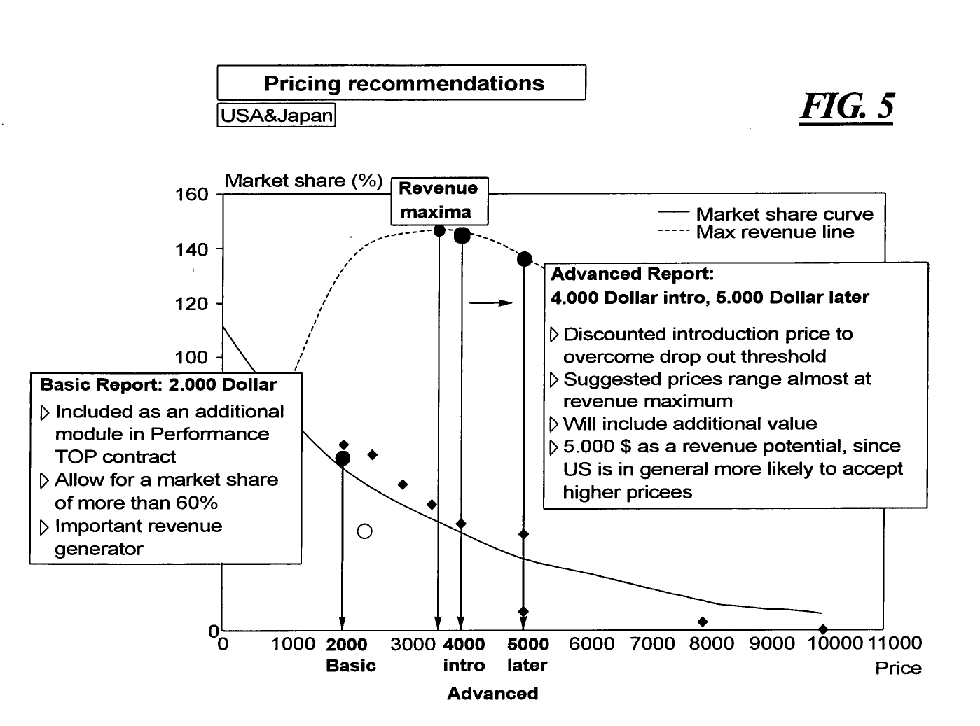

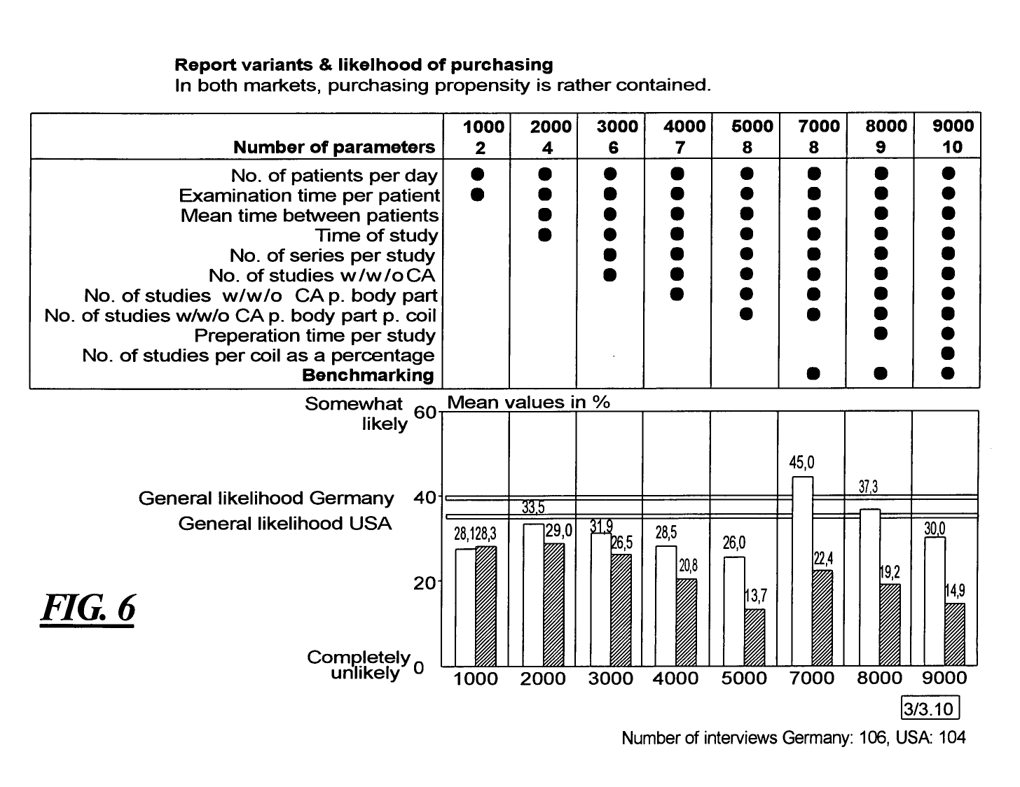

Referring now to FIG. 4, a system workflow browser is also provided according to embodiments of the present invention. The system work flow browser monitors not only the control panel for the computer tomograph (CT) installation, but also other control panels for other devices in the medical facility which are operated by the users. The system workflow browser combines the representation of activities by the users from each of these control panels and thereby accesses the load on the respective devices and the distribution of the function blocks on these devices via monitoring of the control panels. The function blocks in the graphical representation of FIG. 4 are combined according to the examination types (such as a heart examination patient), as illustrated in a navigator portion 74 of the display. Cardiac, thorax, multiple trauma and head examination times are displayed separately, and are displayed as averaged or combined times for the procedures. Three further graphical portions of the display 76, 78, and 80 are provided with analysis of the usage of the various technical units, etc.

The graphical representation of the workflow browser displays the times at which an examination is implemented and the particular panels on which the examination is processed. The workflow browser thus shows the entire workflow of the computer tomograph installation facility in a clear and compressed form. In addition to displaying the duration of the function blocks and the pause intervals, an assessment may also be made as to whether work steps are distributed among the consoles in an effective and efficient manner. For example, it may be recognized from an examination of the system browser that the post-processing is performed on the control panel of the computer tomograph equipment which leads to a delay in examination of the next patient or, it may be apparent that the duration between the examination and the finding or results of the examination may be shortened by additional consoles because the post-processing is performed on the existing consoles and this represents a bottleneck due to overloading of the existing consoles. A result of the examination could reveal that the existing consoles are sufficient in number but are not be optimally utilized, for example, by a lack of personnel. A corresponding scenario may be present in which higher patient through put or shortened working times may be accomplished by adding personnel.

As noted above, a benchmarking engine is provided which accesses data that has been supplied to a central customer workflow data bank in which a number of customer profiles are contained. The customer profiles include the demographic data relating to the particular clinic or customer being monitored according to the present method. Workflow data is recorded for each customer or clinic. These workflow events are triggered by the software and include the following parameters which are stored in a log file with a time stamp. The parameters include, patient positioning, which includes positioning of the patient on the computer tomograph apparatus and then moving the patient from the computer tomograph or assisting the patient in getting up from the computer tomograph apparatus. Further information logged in the workflow data includes the average operating time of the scanner noted in detailed per work step. This includes patient registration, examination protocol selection, examination preparation, examination planning, scanning or eradiating of the patient, protocol specific reconstructions, examination conclusions, protocol-specific examination durations, or protocol-specific number of images per examination. An additional item of workflow data includes the ratio of post-processing to examination times, which may be sorted per each examination type. A further items of information recorded may include minimum and maximum examination times which may likewise be sorted per examination type. The consumption of materials utilized in the examination, such as the quantity of contrast agent administered to the patient is likewise recorded. Another important item of information recorded in the present method is the duration of time in which the device is not being used. This may include the time from the end of the scan until the start of a reconstruction, the time from the end of the scan until the conclusion of the examination, the time from the conclusion of the examination until the next patient is registered, etc.

Yet another workflow data item recorded is the number of actions or events performed during the work flow. These include the number of scan terminations by the operator, the number of the protocol selections from the protocol list, the number of changes to the examination parameters, the number of mouse clicks (or other point or device activations) in the selection of the examination protocol, the number of mouse client (or other pointing apparatus activations) for reconstruction jobs, the number of automatically or manually started reconstructions, the number of reconstructions per scan range, the number of thin slice reconstructions (which may be up to 1.5 millimeters in thickness, the number of images per examination or scan range or reconstruction job, the number of automatic transfers per scan range, the number of images per automatic transfer, the number of automatic recordings per scan range, the number of images per automatic recording, and the number of loaded images per computer tomograph application.

Further, data items recorded in the workflow analysis include the frequency of examination protocols used, the frequency of temporary adaptations of standard protocols, the frequency of automatic patient instructions utilized (such as breathing commands for each type of examination), the frequency of automatic or manual reconstructions, the frequency of automatic transfer, the frequency of automatic recording, the frequency of use of the applications, and the frequency of non-examination specific functions per examination (these may include viewer functions, recording functions, 3-D image functions, browser functions, etc.

A further item of information includes statements concerning the capacity of the various devices in the clinic facility. For example, the load on the computer tomograph scanner is recorded, along with the load on the various control panels, the load on the workstation, the load on the Leonardo and the load on the network or public access computer system.

Thus, by an evaluation of the workflow browser displays as shown in FIG. 2-4, the weak points and bottlenecks in the system may be identified by comparison to best practice users. A consultation talk may address the results together with the customer and plan modifications in the process steps. The customer or clinic may follow the recommendations and then may compare the success of these recommendation by performing a later benchmarking for comparison with its earlier operation.

The workflow analysis according to the present invention provides various advantages. Among them, is a transparency in clinical workflow. The workflow report is comprehensive and clearly informs the users of the workflow. Four different representation forms are automatically generated according to a preferred embodiment. In particular, a short report is generated to provide an overflow of the workflow in the form of tables with statistical evaluations. This short report form serves as an introduction to the analysis being performed in the workflow. A standard representation is to show the standard workflow analysis by combining the data from a plurality of examinations taken over an arbitrarily set observation time span. Such time span may be one month, one day, one week, etc. A standard evaluation is performed and the display of averaged function blocks is provided in contract to the workflow event browser. This form of representation is particularly well suited to implement the analysis steps of comparing different examination types.

A third representation according to the present invention is a workflow event browser. This shows the work flow at predefined points in time. This representation helps to provide a targeted analysis of specific examination steps. The analysis performed on the standard workflow may be analyzed exactly by looking at the potential for improvement and identifying such potential.

A further representation is that of the system workflow browser, which shows the entire workflow for a computer tomograph installation in an overview form. The load and the distribution of function blocks on the various consoles can be readily assessed and optimized.

The types of reports which are provided according to the present method are optimally complementary to one another and provide a total overview image of the workflow. Improvements may be readily made which help the customer to optimize the workflow himself or to implement the optimization with the support of a consulting service.

An aspect of the present method is to provide individual feedback to the clinic or user. The data acquired from the workflow analysis allows conclusions to be made about the efficiency and operating performance of the customer or clinic. The customer or clinic may have individual functions reproduced via the display in an objective manner. In addition to being provided with the workflow report, a consulting service may be called to assist the customer or clinic in identifying erroneous usage or sub-optimal work processes and in recognizing and implementing common possible improvements (such as software or hardware upgrades or restructuring of personnel assignments.

A further aspect of the present method is to provide benchmarking. The customer or clinic may evaluate the workflow using various criteria found in the benchmarking report. The benchmarking report identifies the position of the particular customer or clinic in comparison with other similarly situated clinics, and may be able to implement improvement through such comparison. Clinics which have an optimal workflow (referred to as best practices) may be identified as desired.

A further aspect of the present method is to provide sharing of knowledge concerning the workflow for the computer tomograph. Contacts are arranged with existing installations of computer tomograph apparatus. A clinic or customer obtains information about the workflow practices of others, assuming approval for such contact is obtained.

FIG. 5 provides information on the value of magnetic resonance and computer tomography (MR/CT) utilization management, showing that significant value may be realized by improving use of this expensive equipment. Parameters analyzed according to the present development have value as well, as demonstrated in the graphs of FIG. 6.

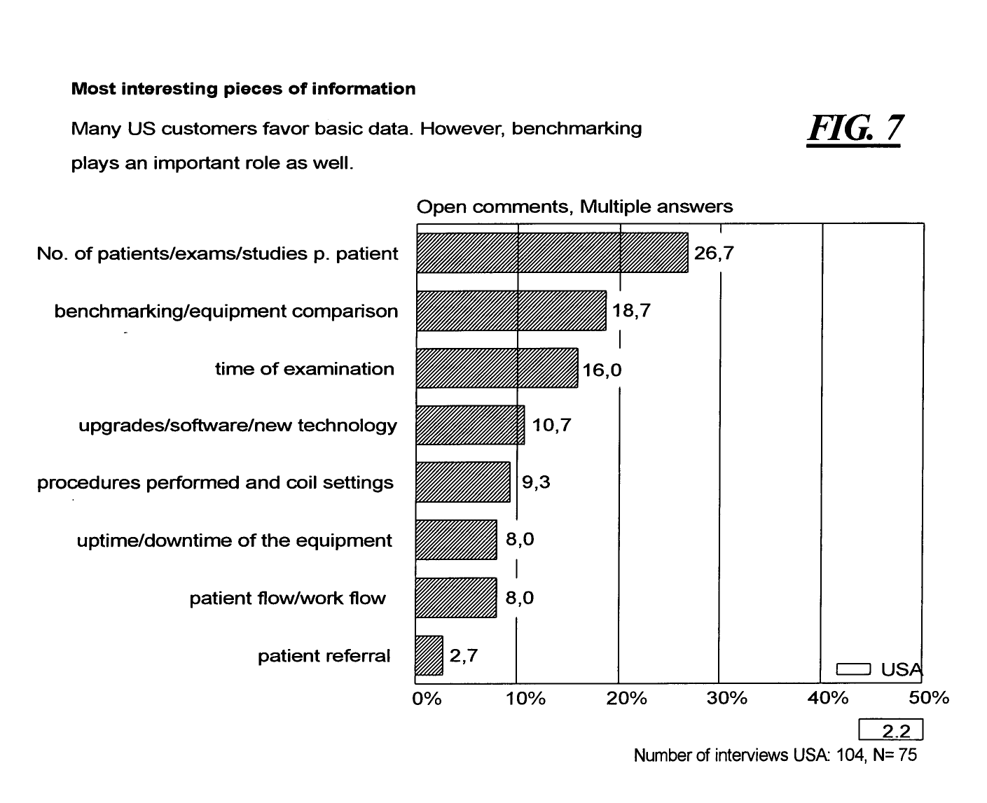

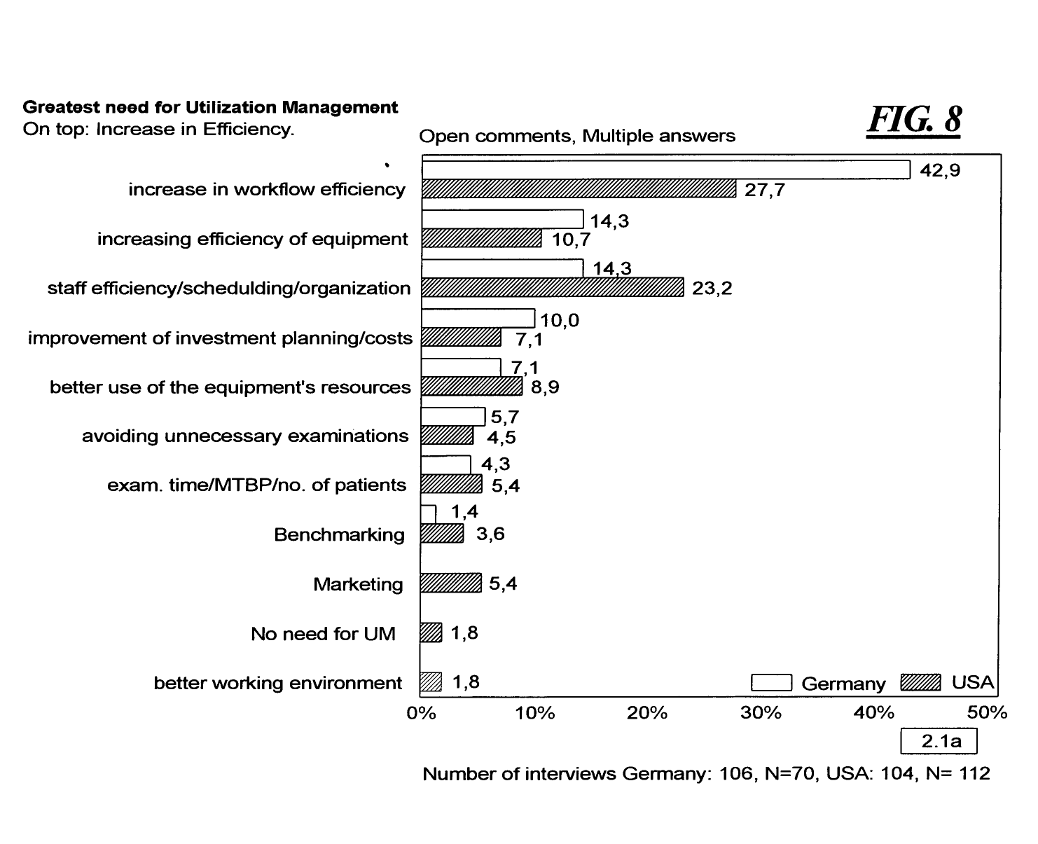

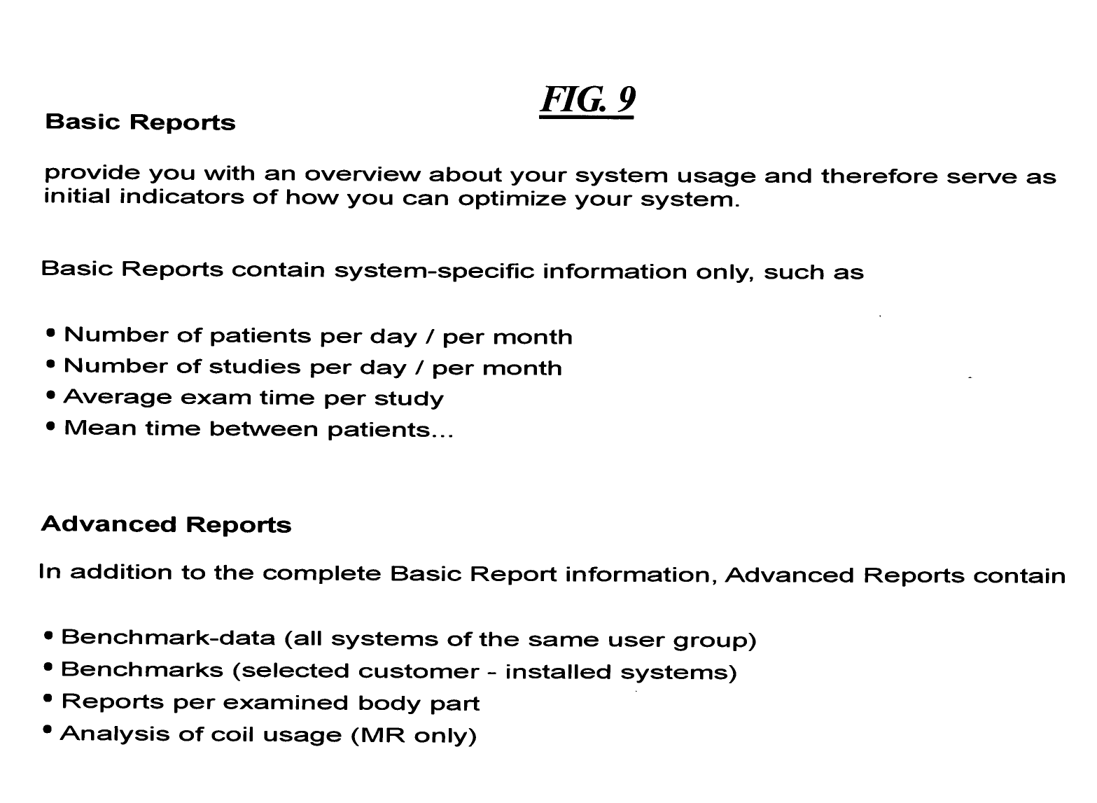

A further advantage of the present method is available to the manufacturers of the computer tomography equipment. The possibility is provided to acquired targeted data about the workflow practices of a customer or clinic. With this knowledge, an equipment manufacturer may offer a customer the support for workflow optimization in the form of consulting services in the field of workflow and applications. An additional business option is to provide scalable usage fees for an expanded utilization management business report. By studying the cost effectiveness for the utilization management using the present reports, other things may be revealed about the customer or clinic which is of interest since the clinic workflow and analysis of improvement potential is transparent. Referring to FIG. 7 and FIG. 8, a comparison may be made between the most interesting pieces of information to clinics or customers (as shown in FIG. 7) and the greatest need for utilization management in the operation of the clinic.

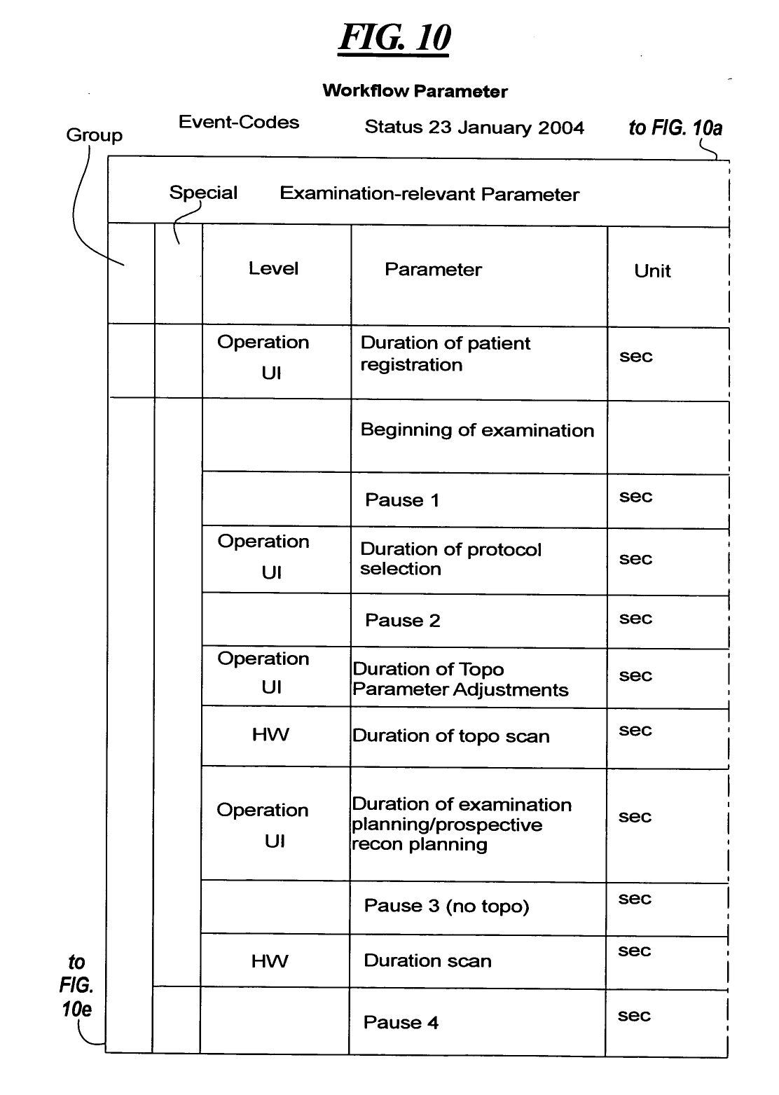

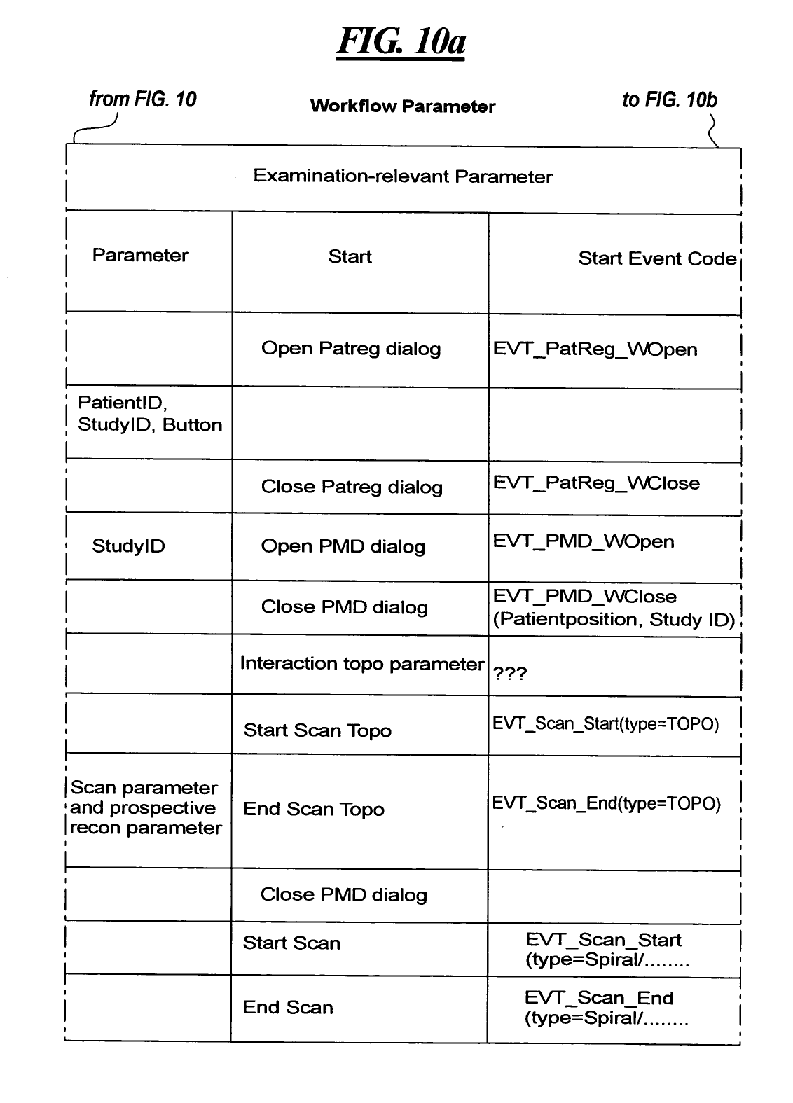

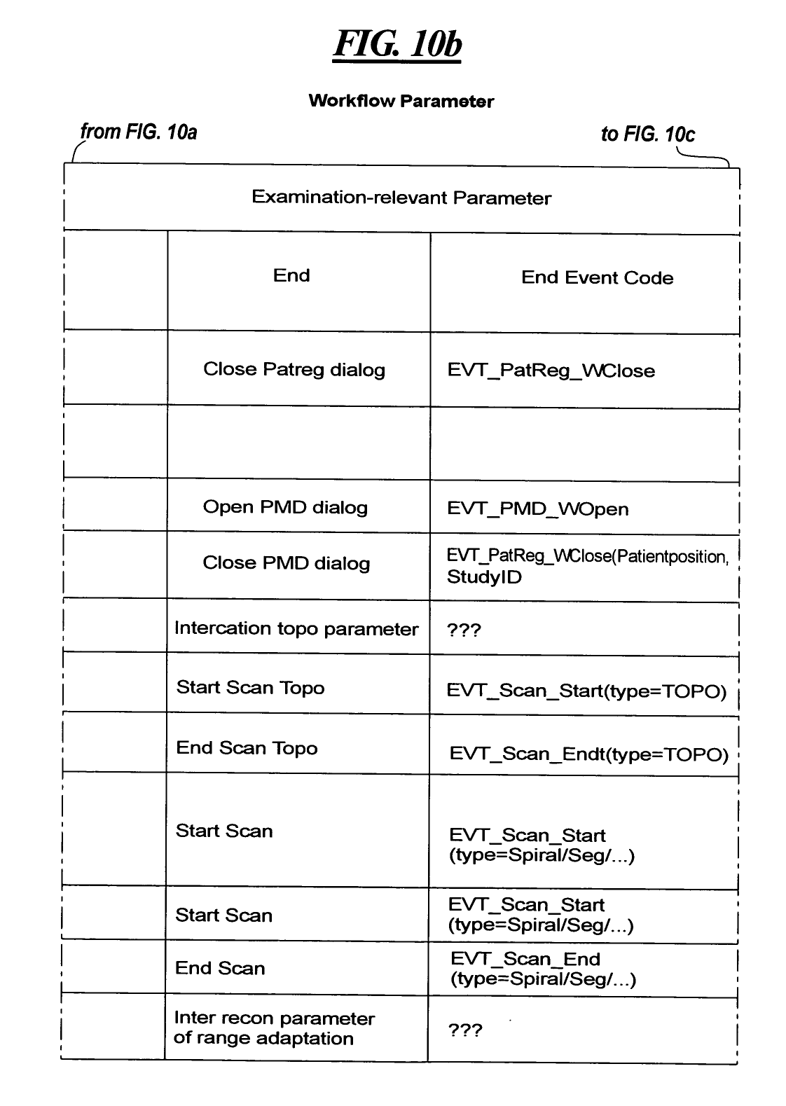

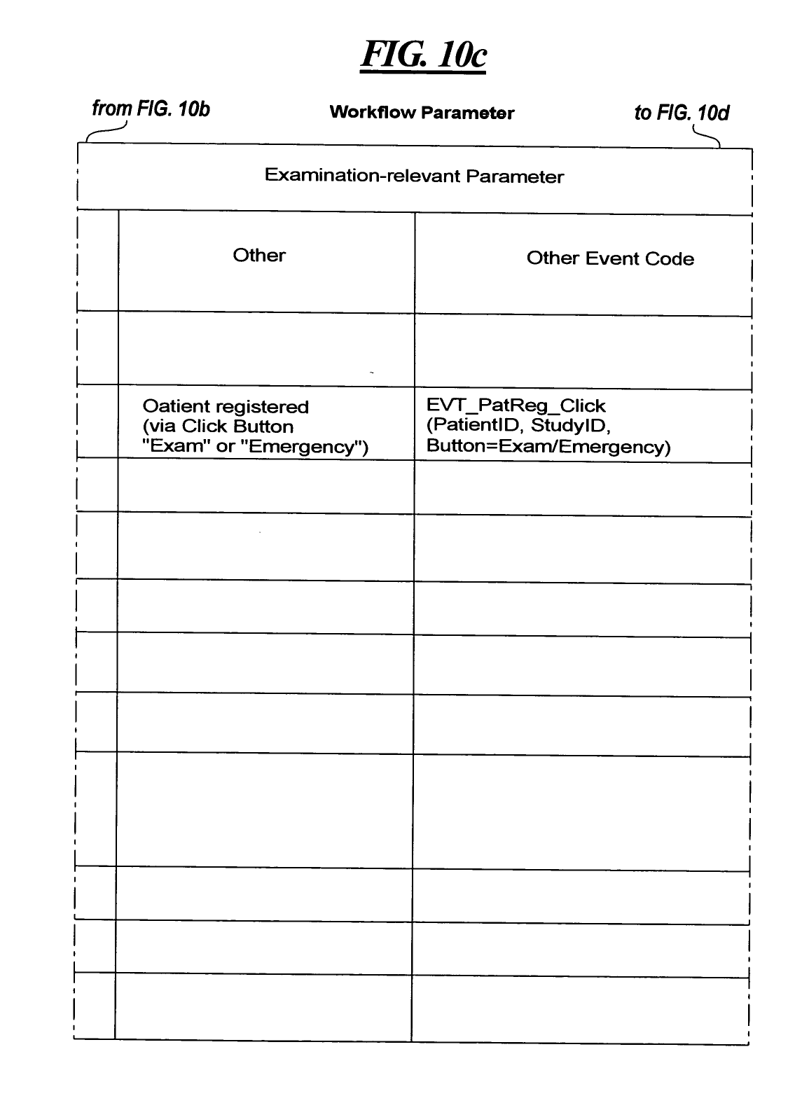

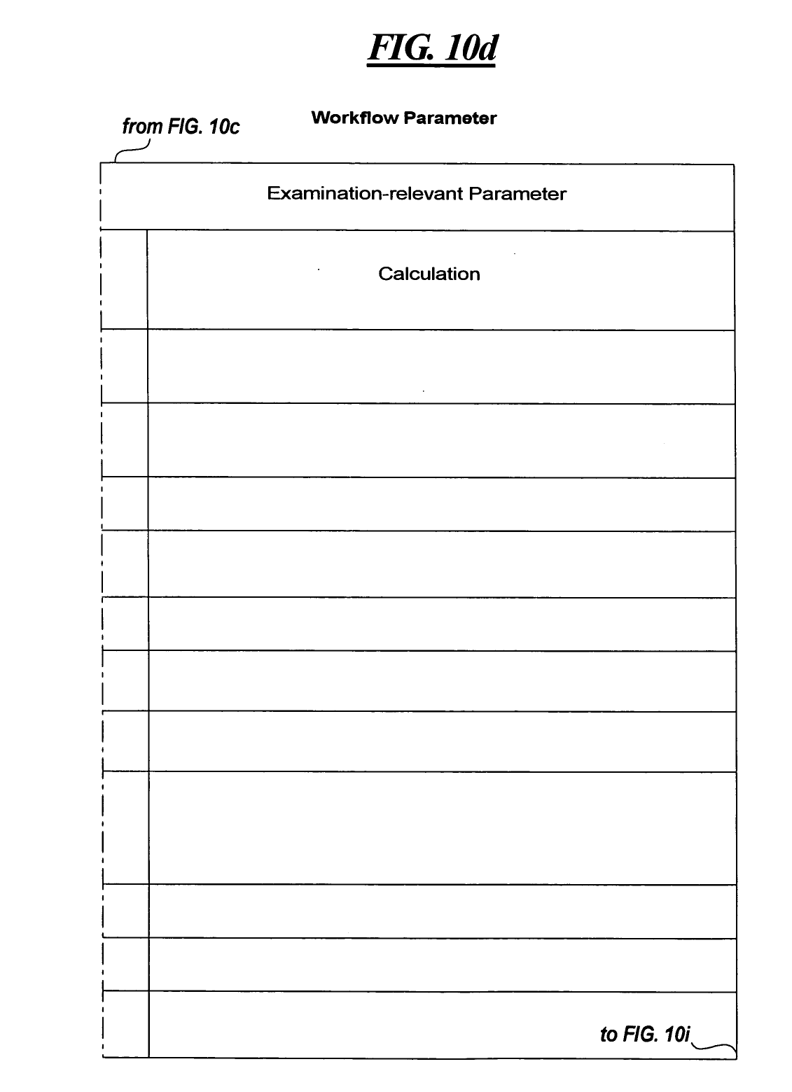

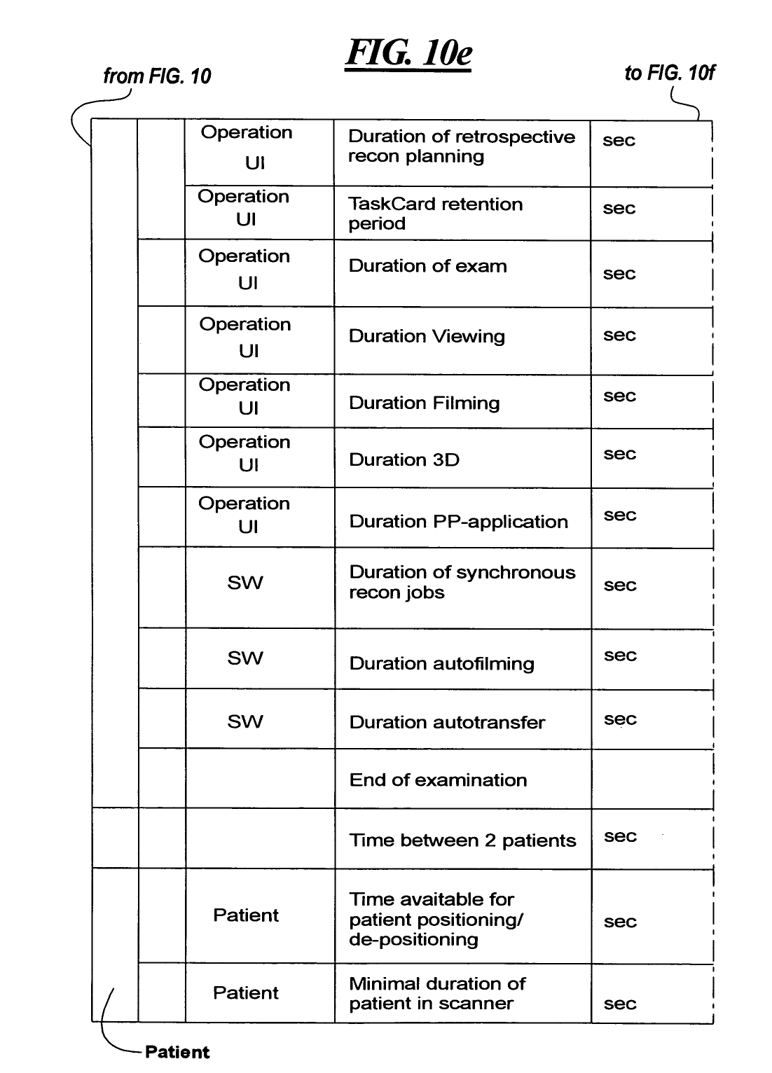

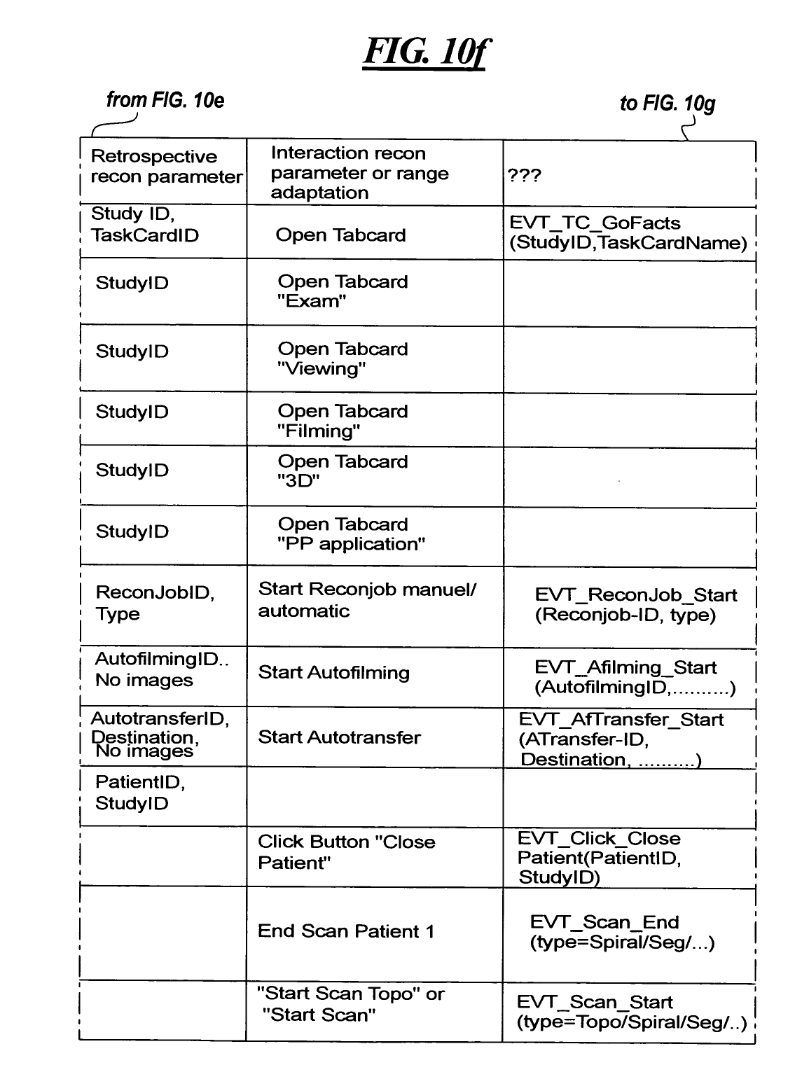

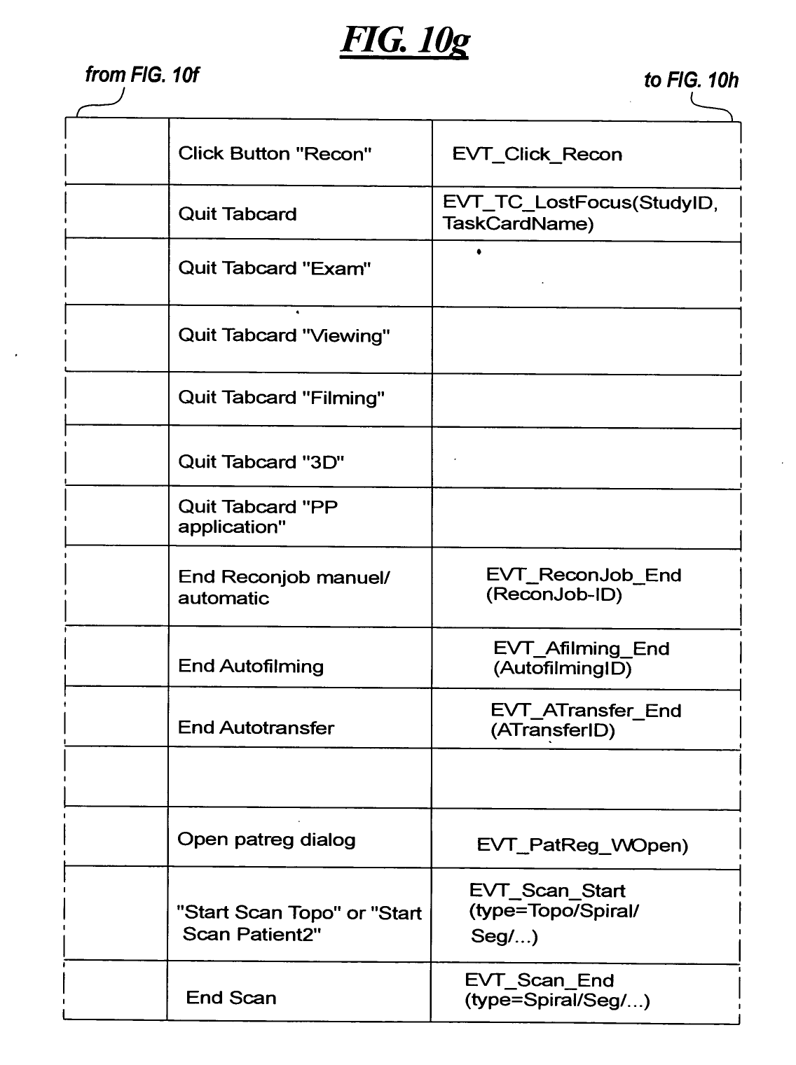

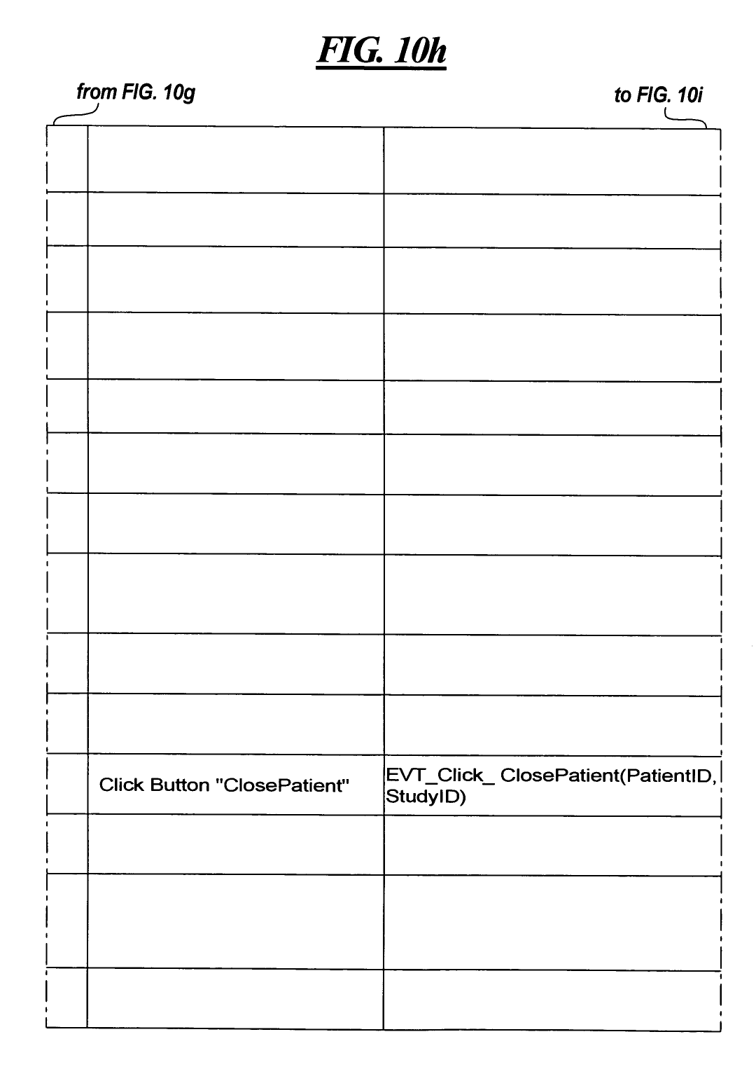

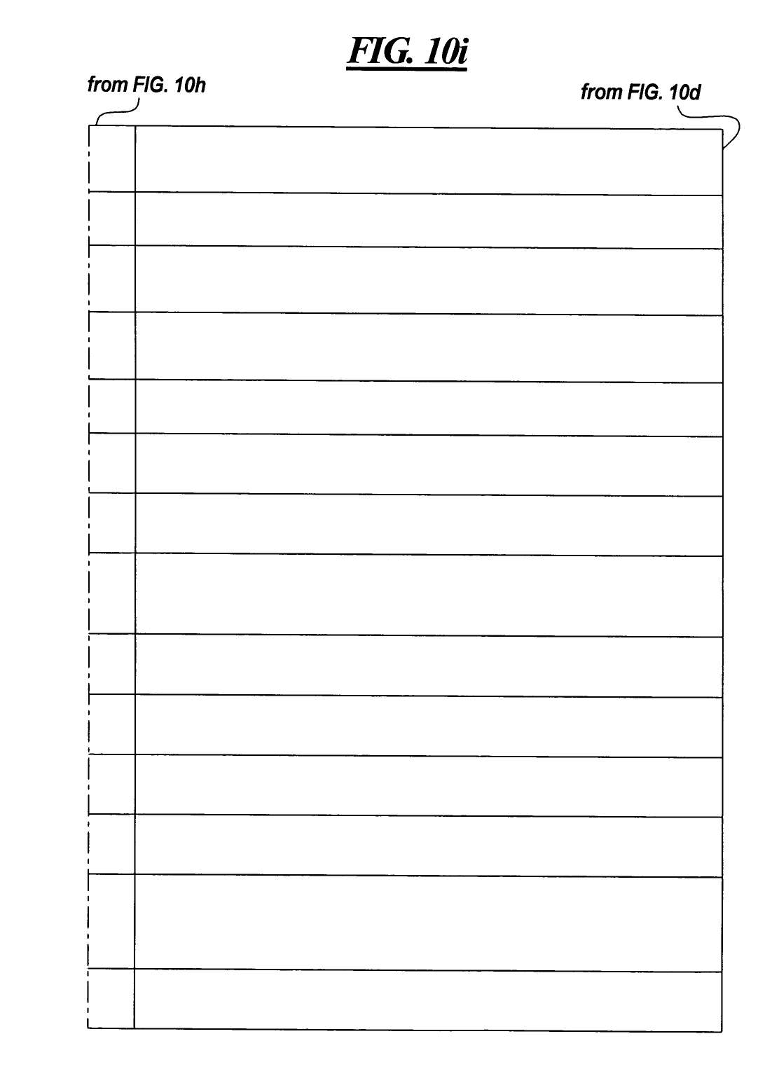

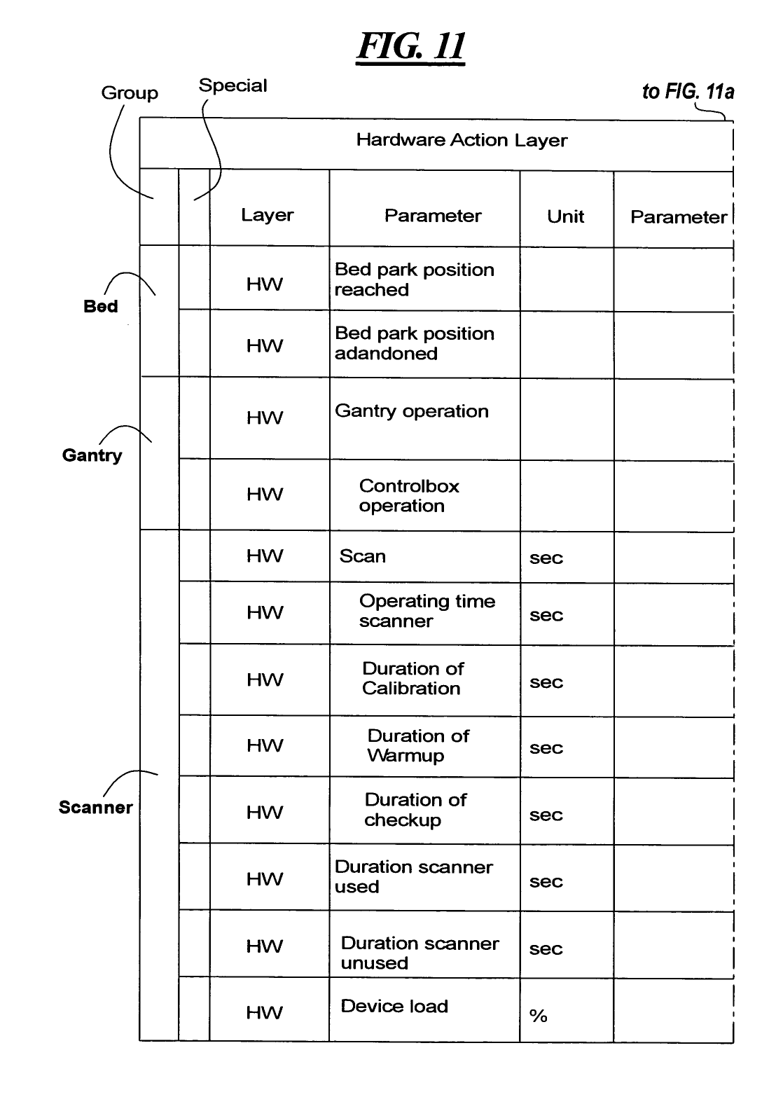

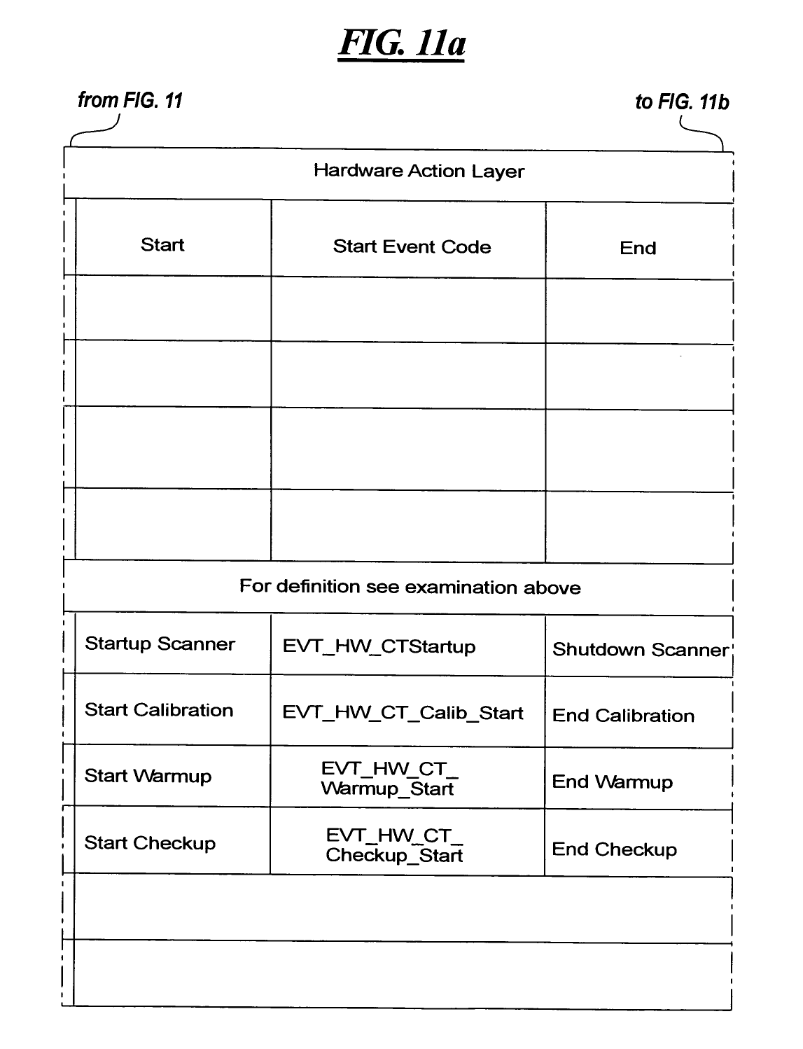

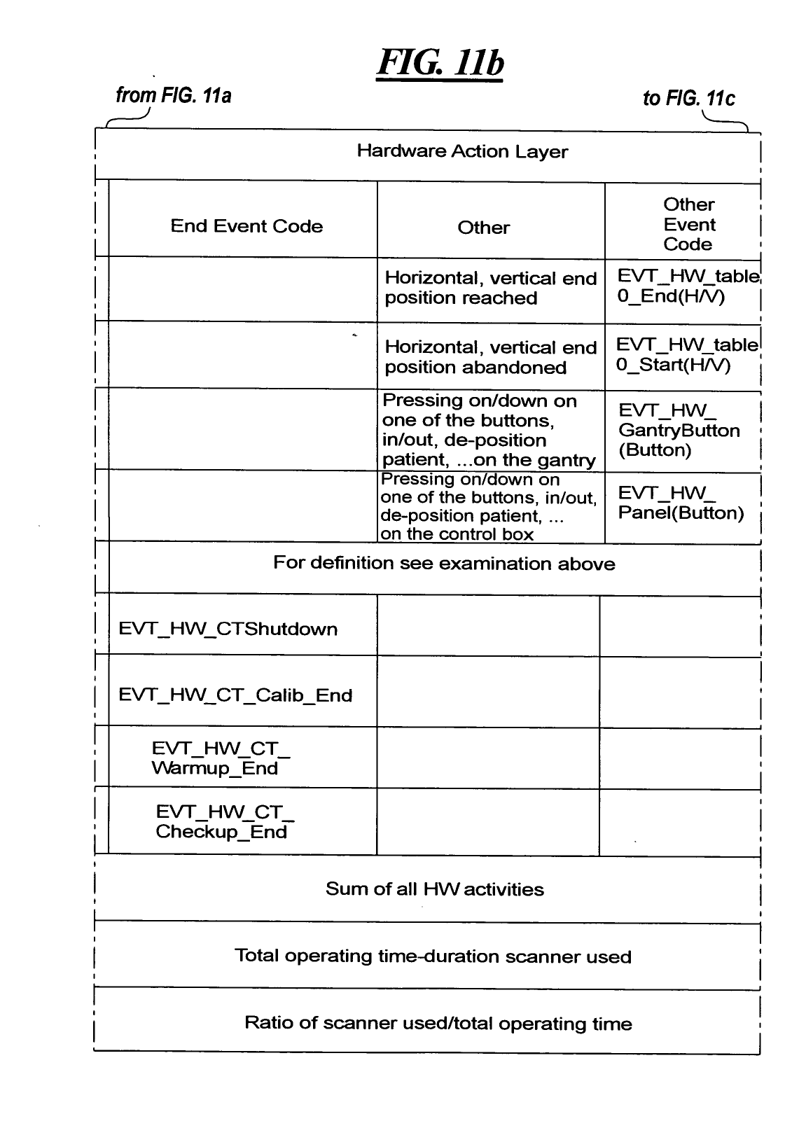

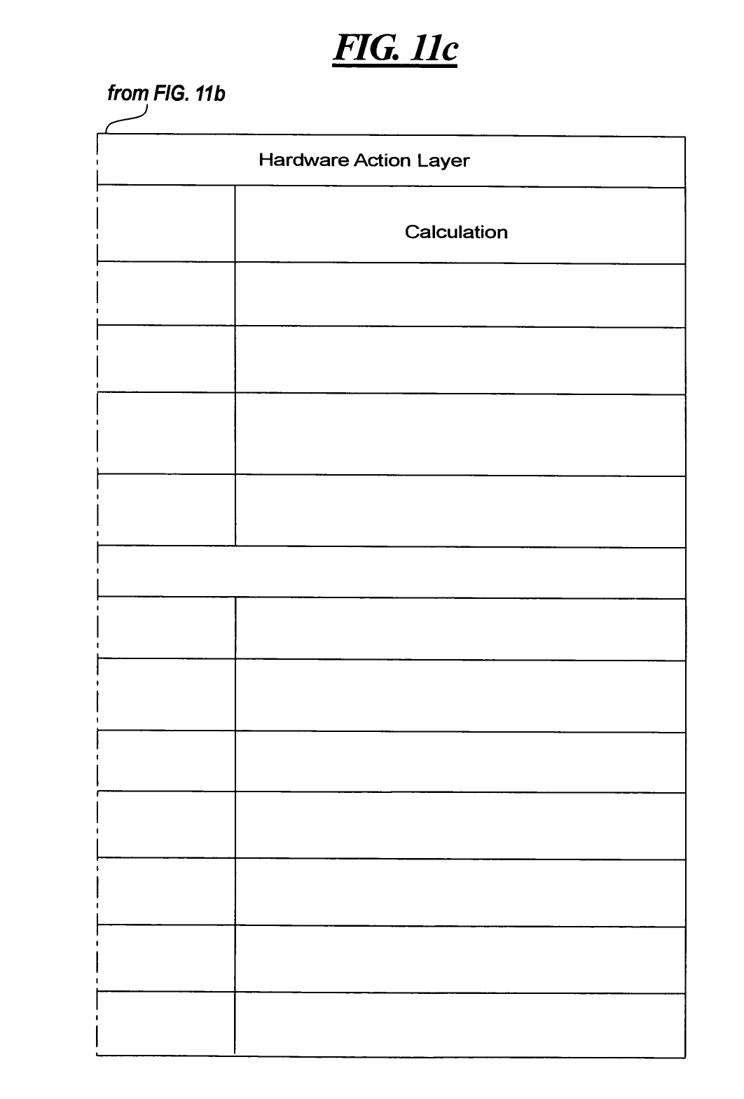

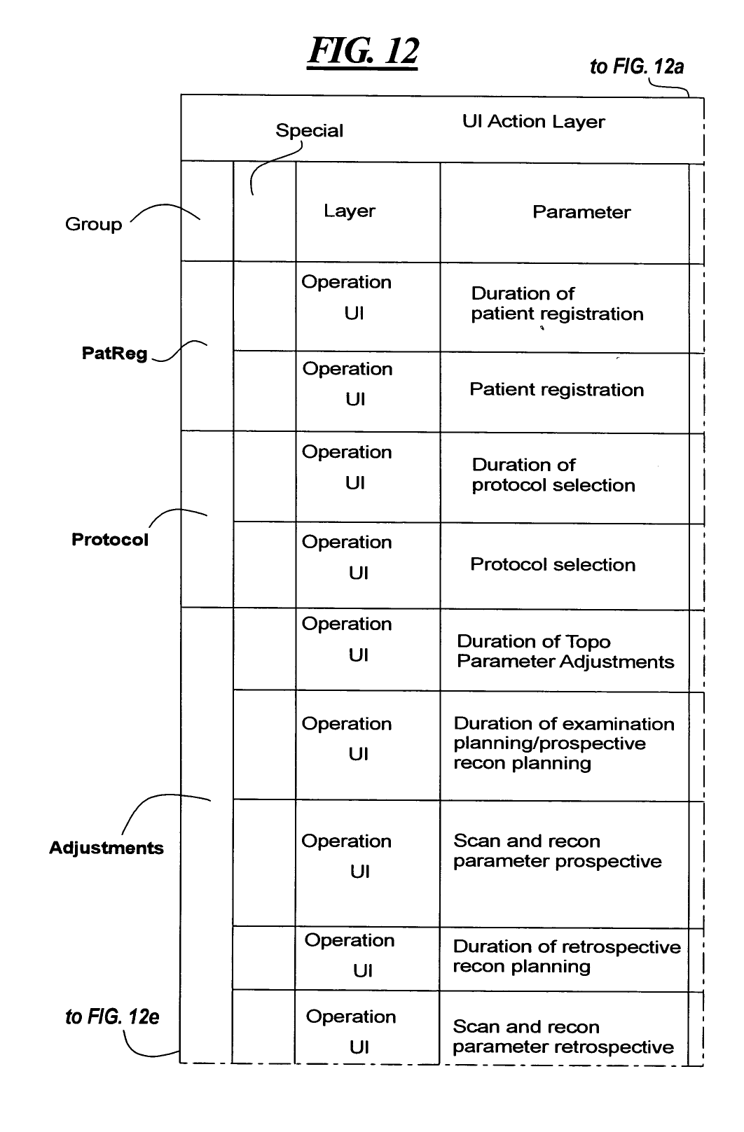

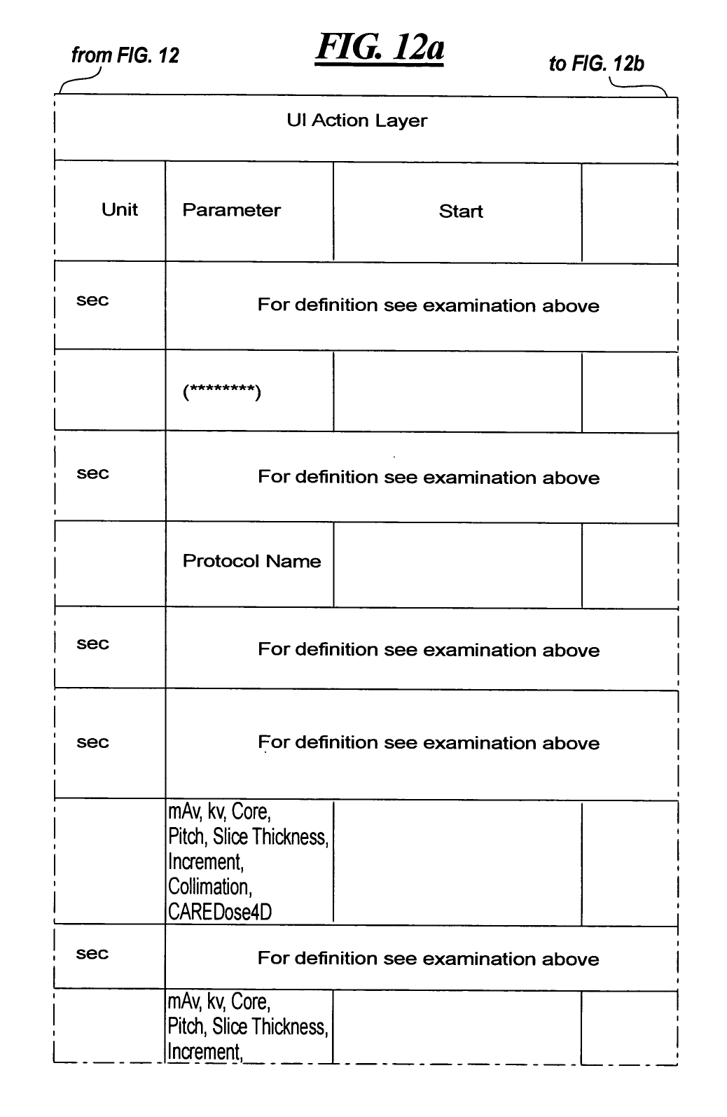

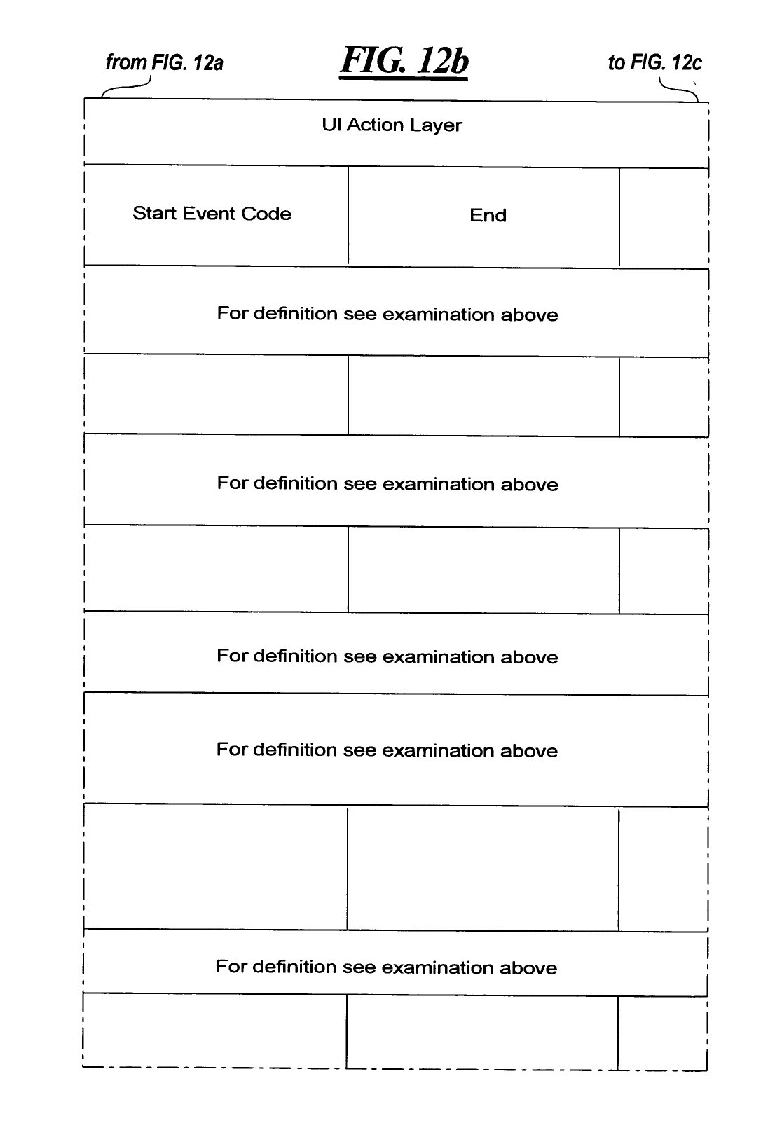

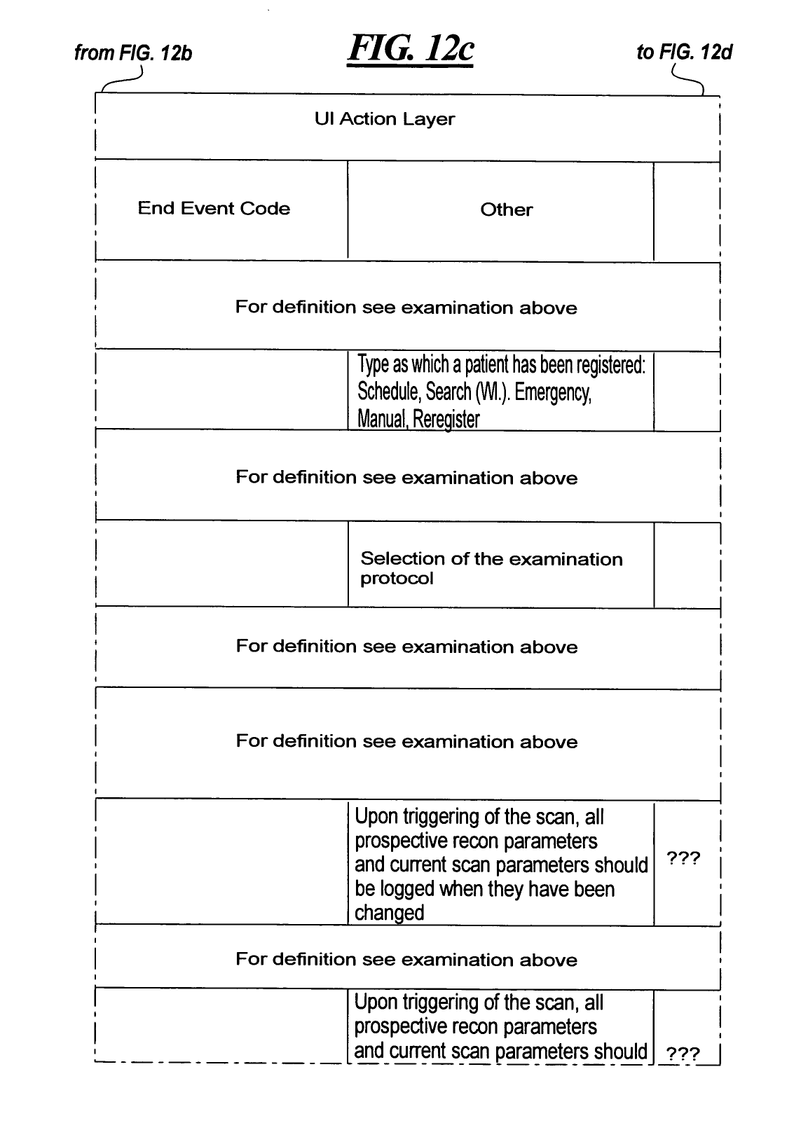

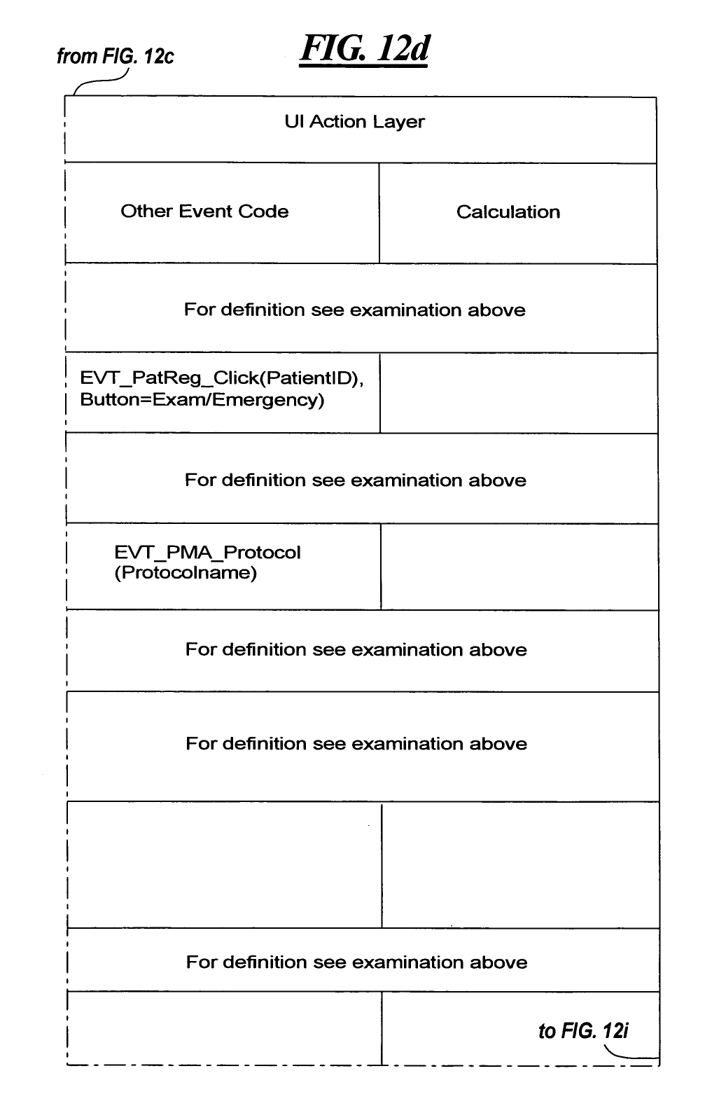

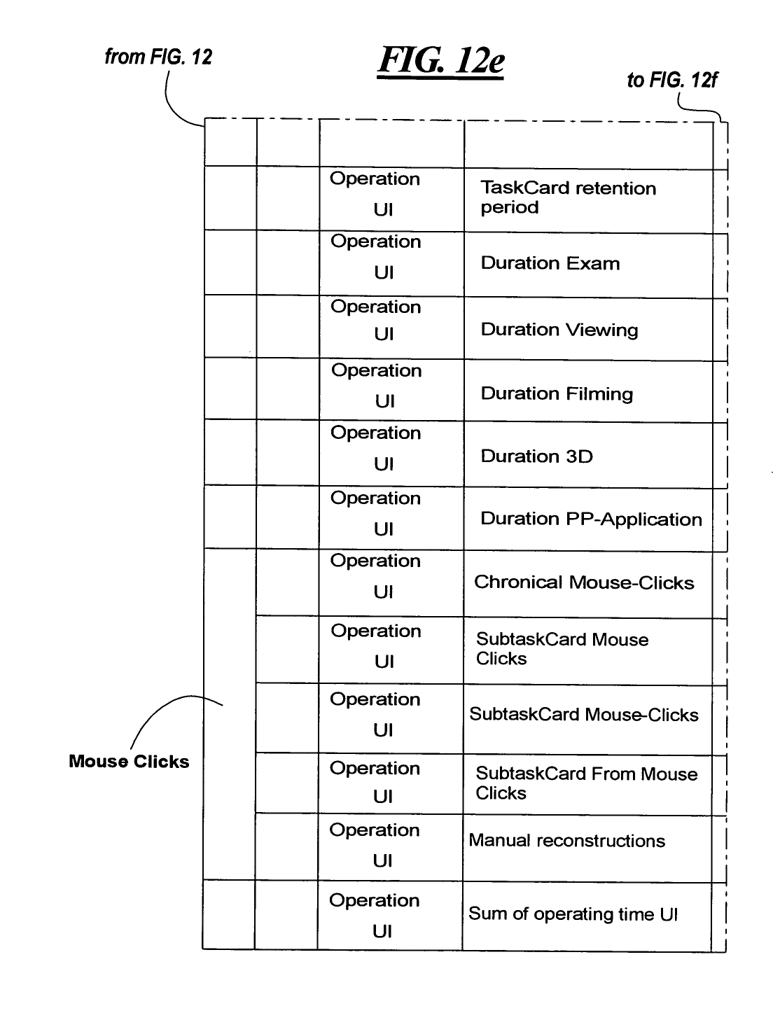

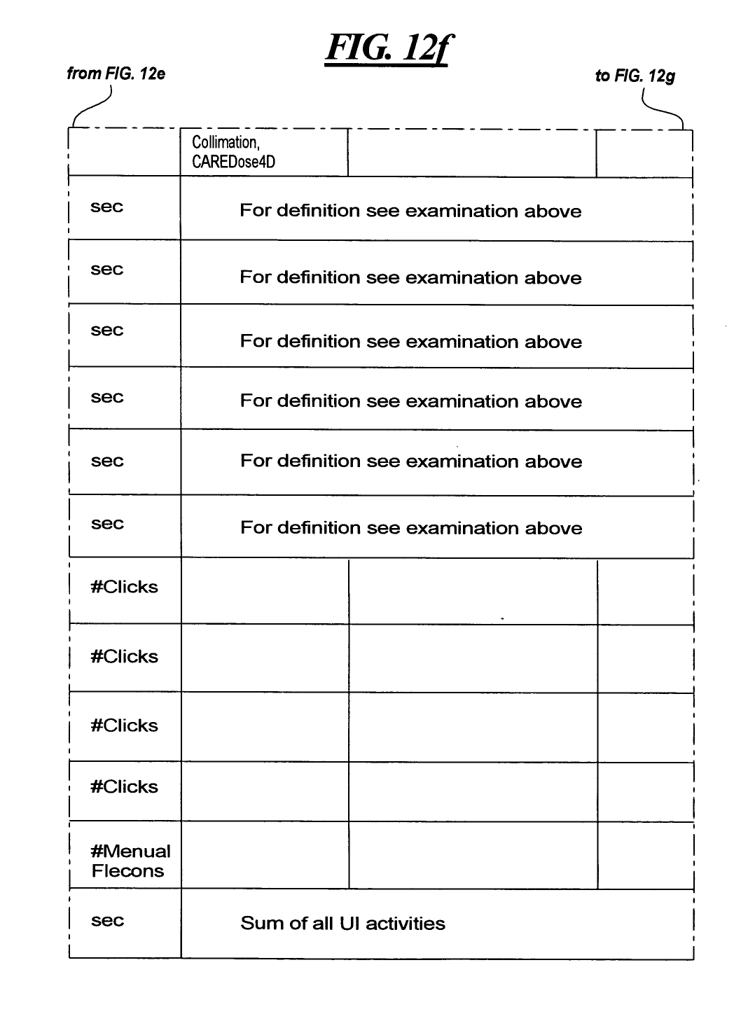

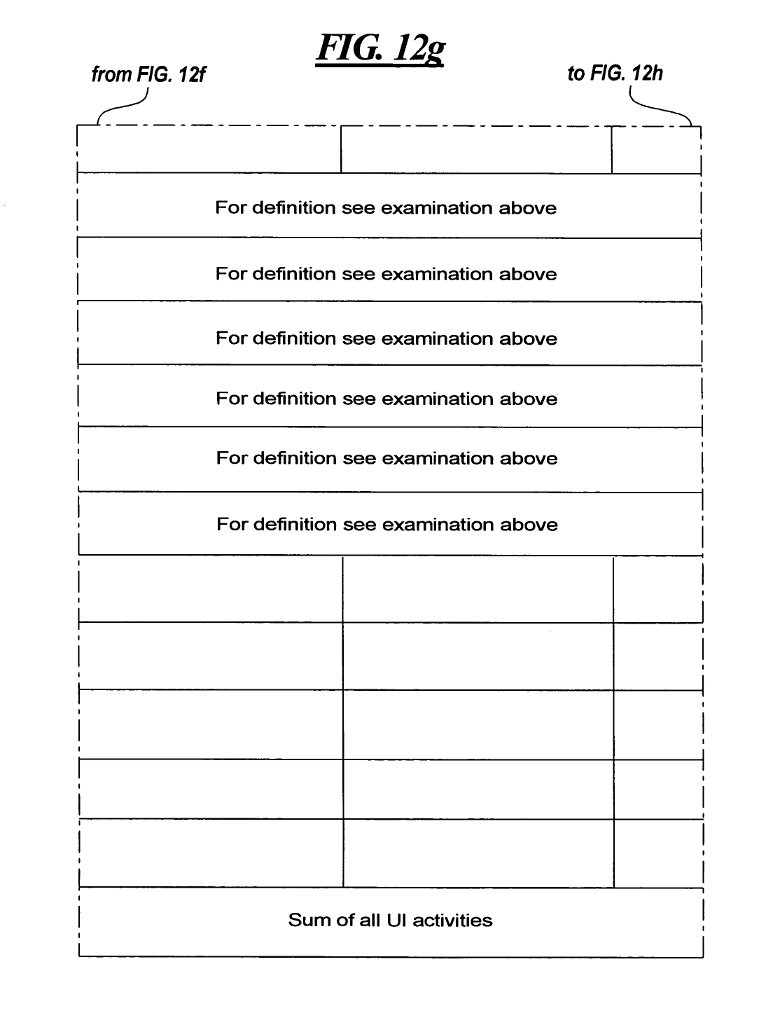

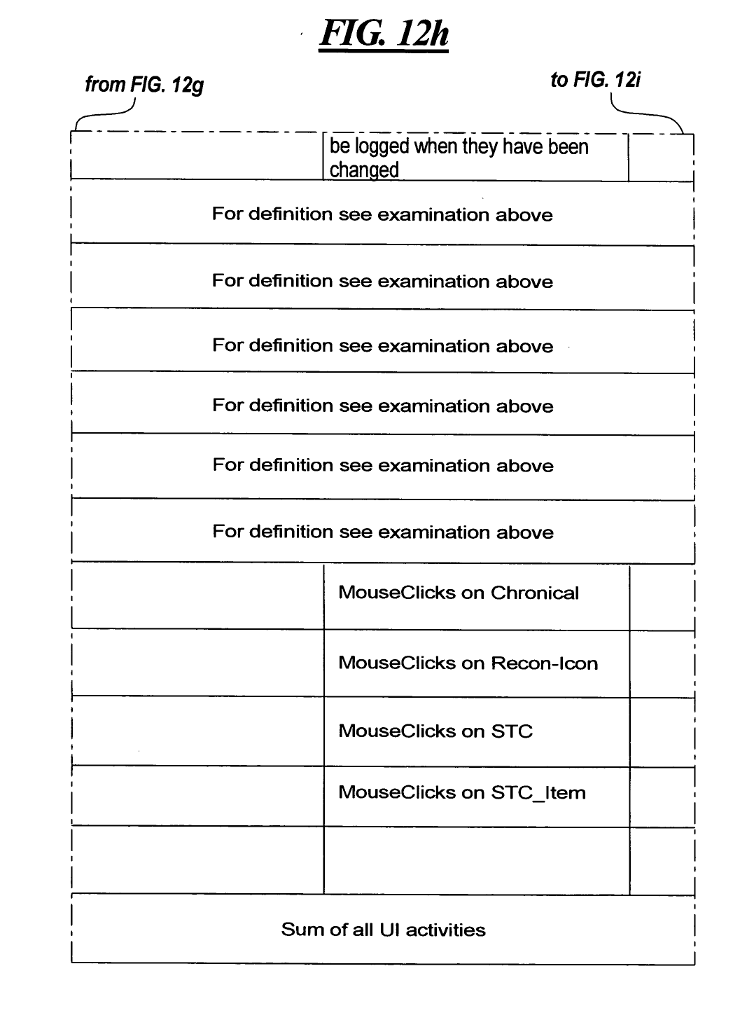

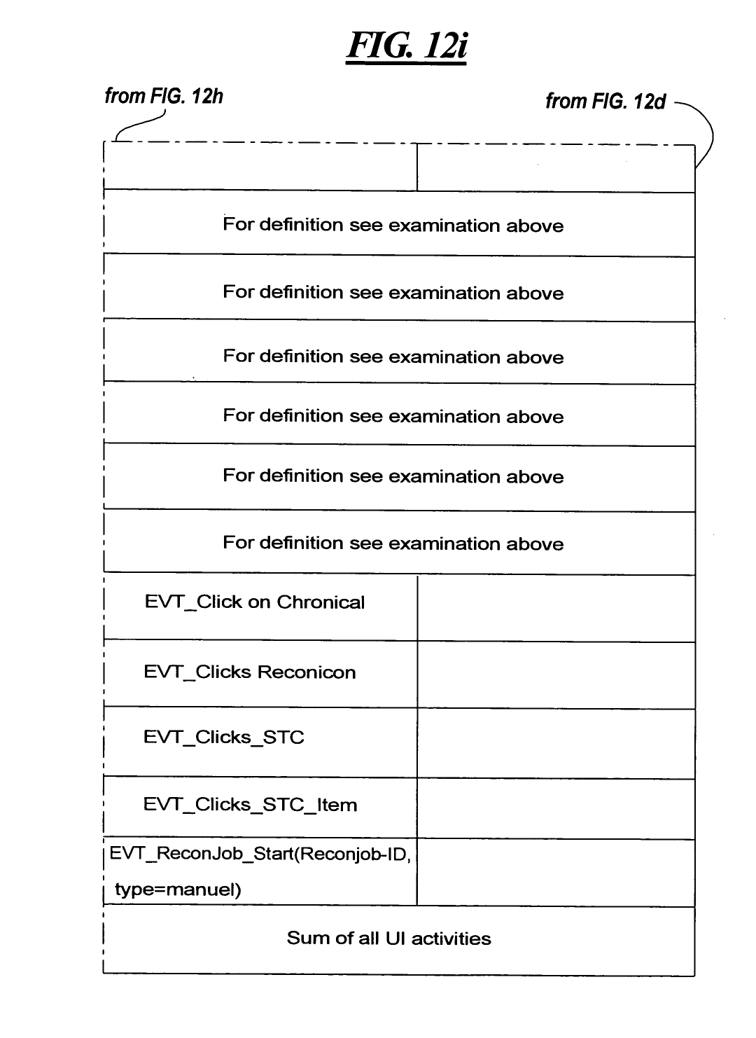

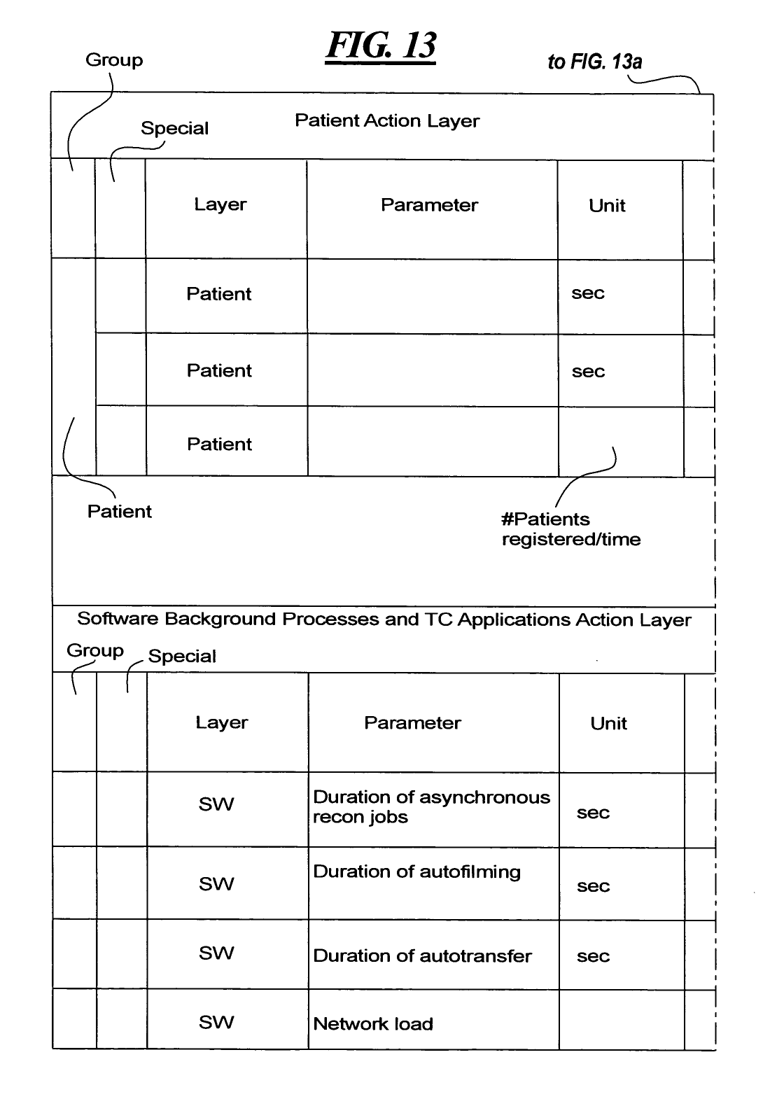

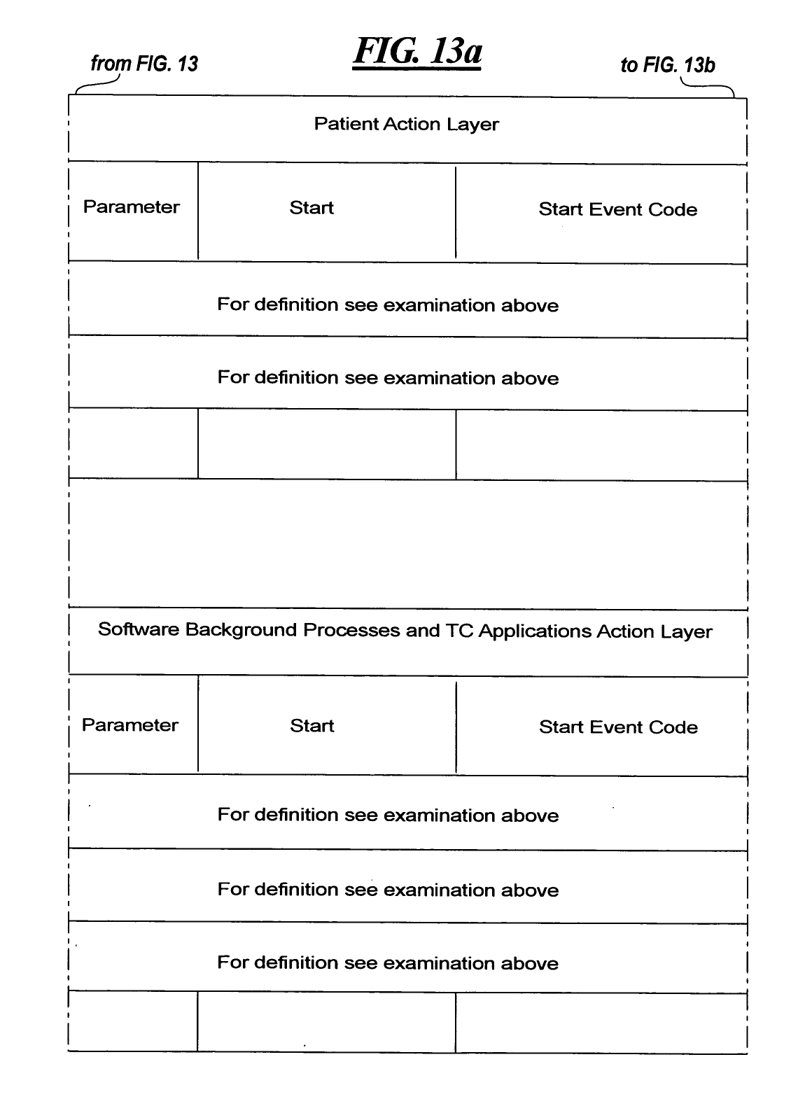

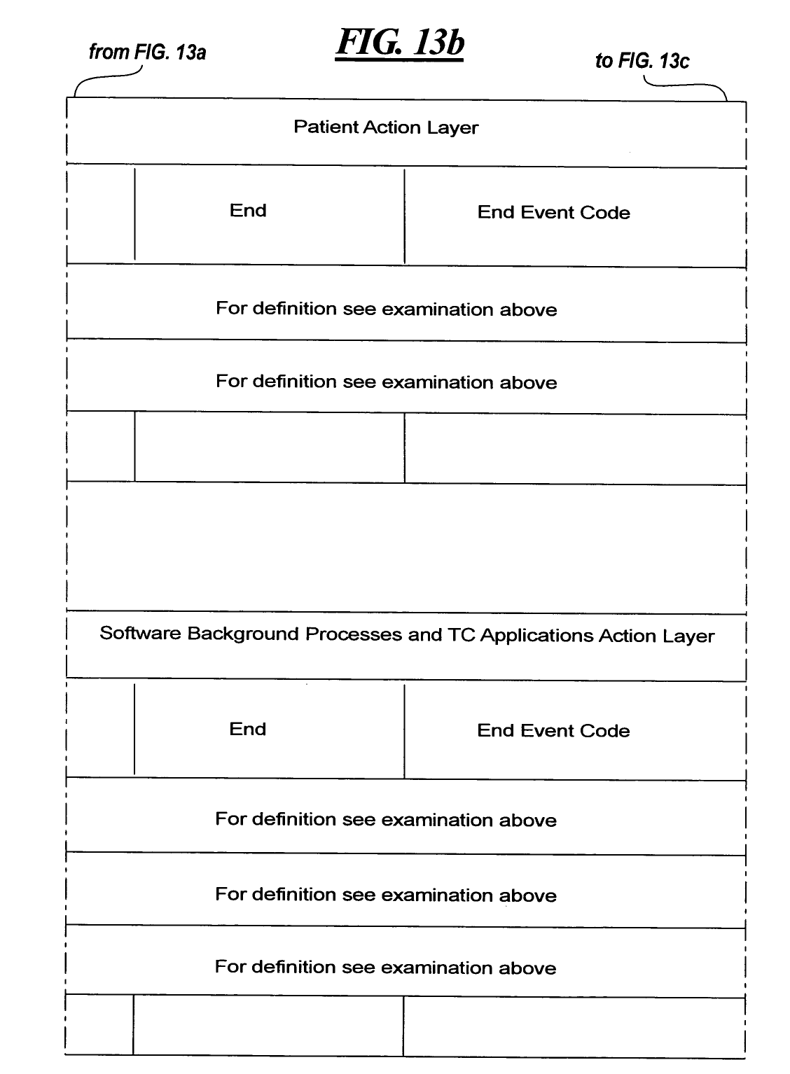

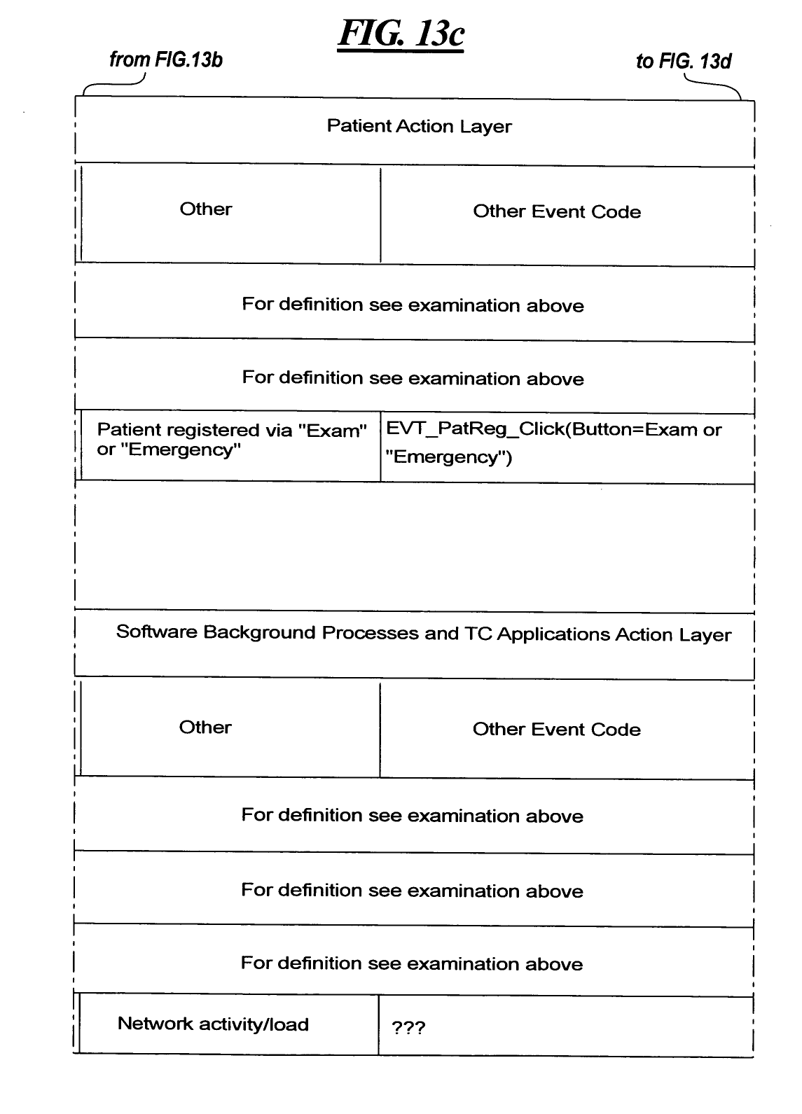

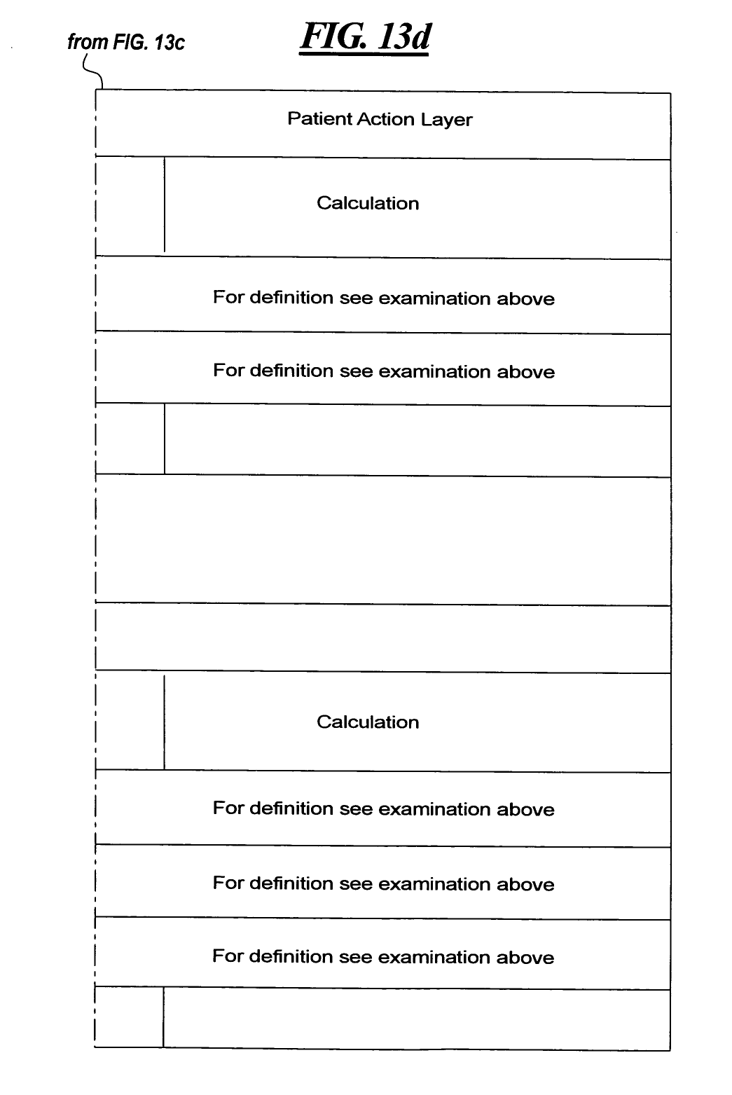

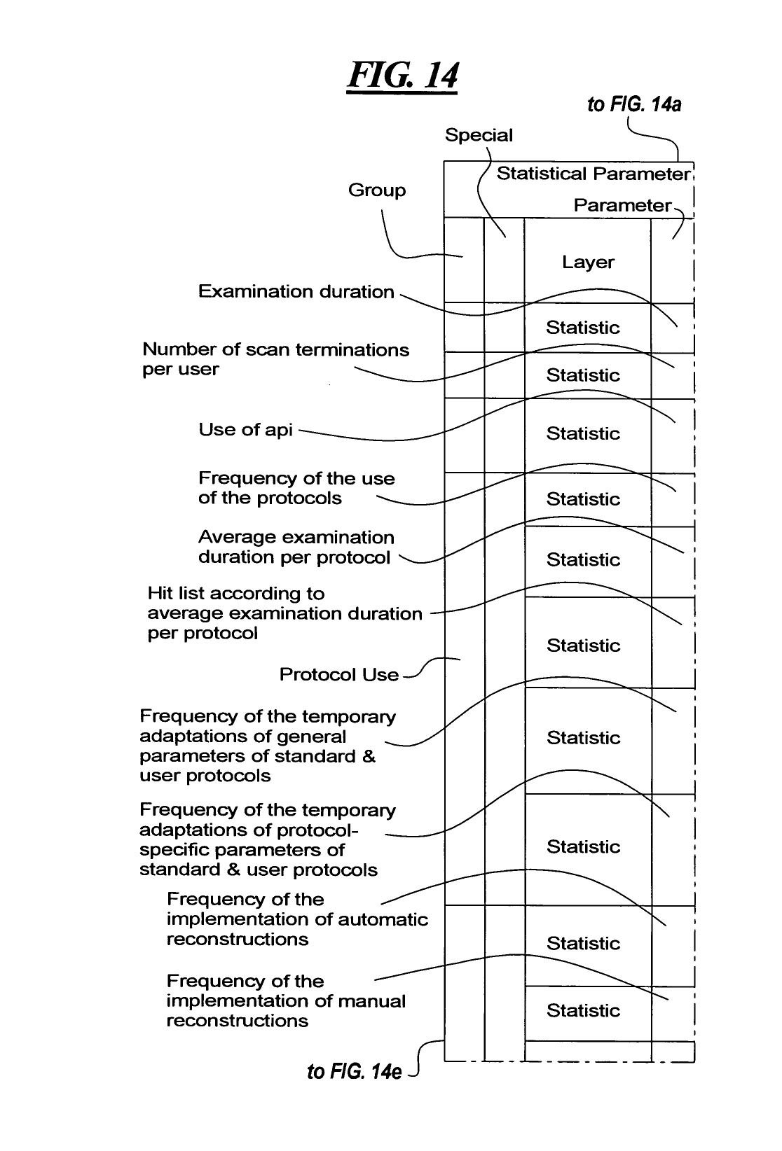

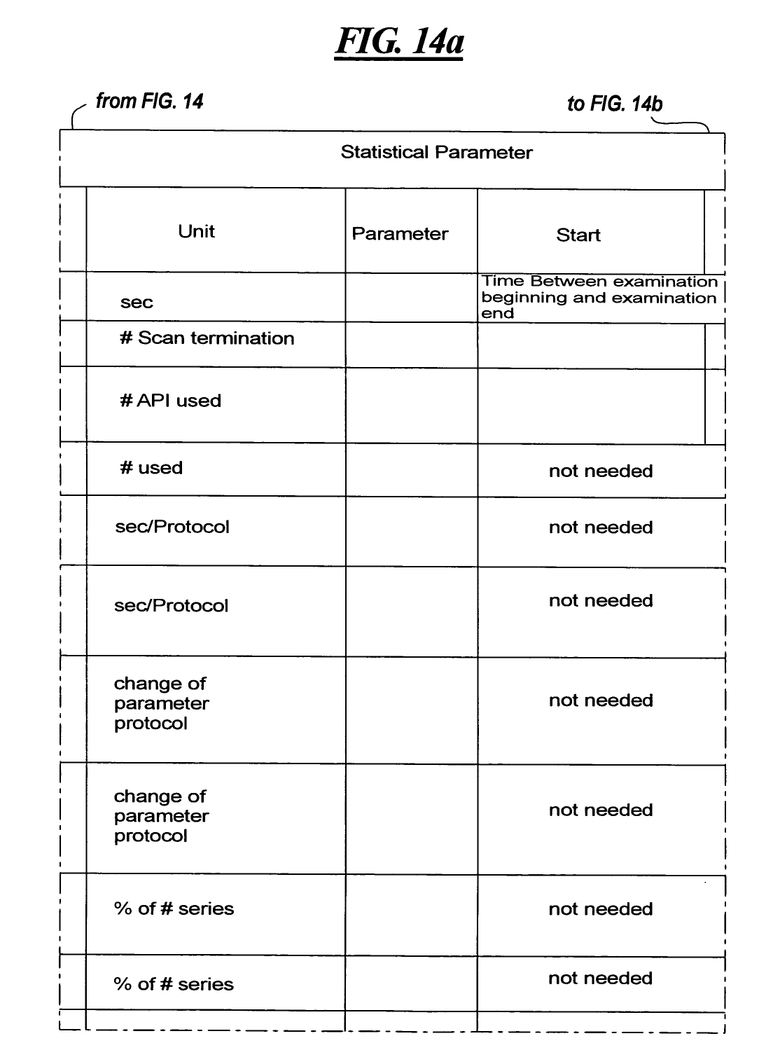

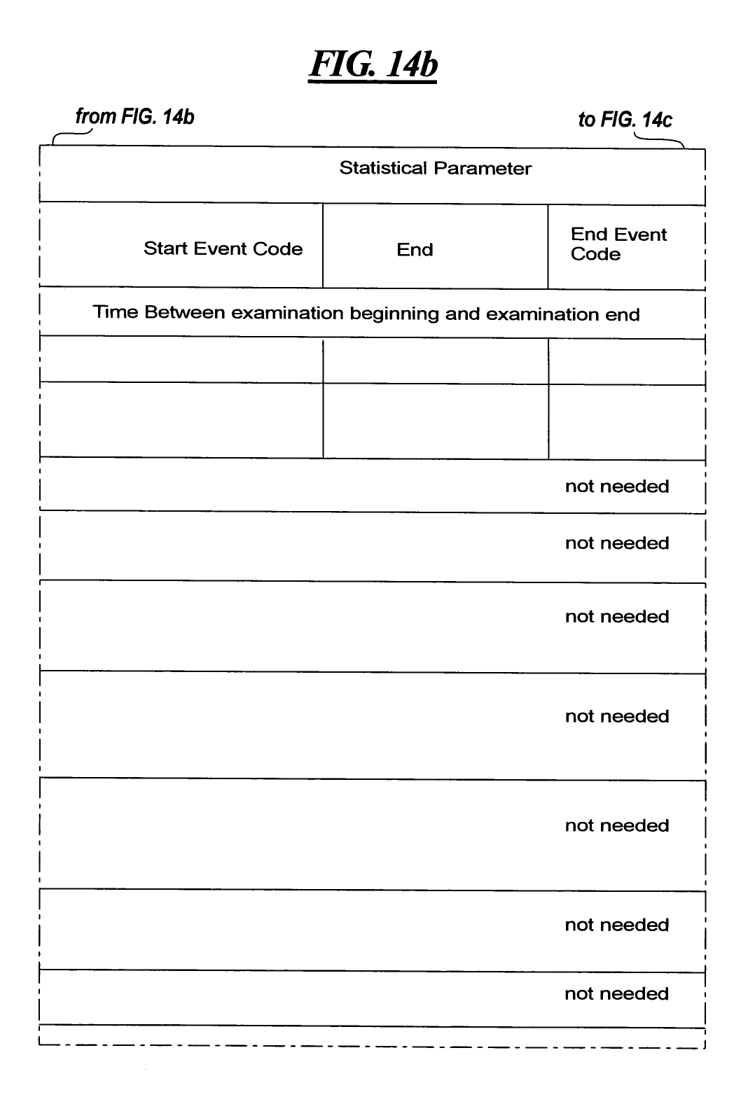

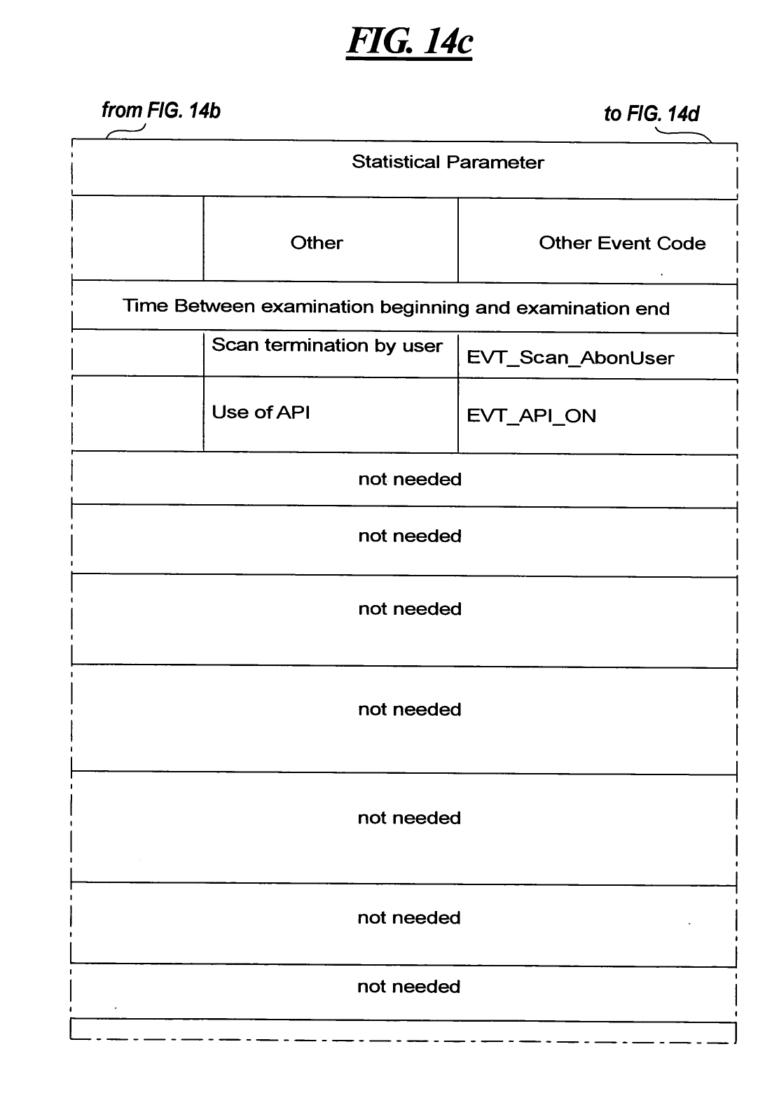

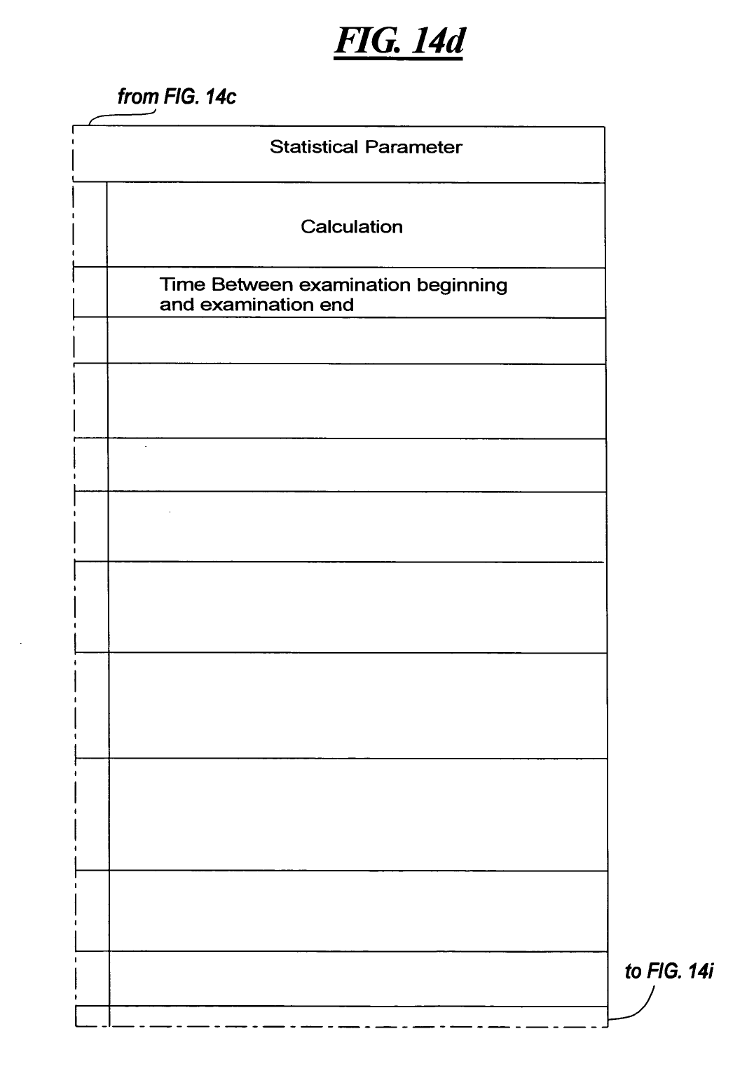

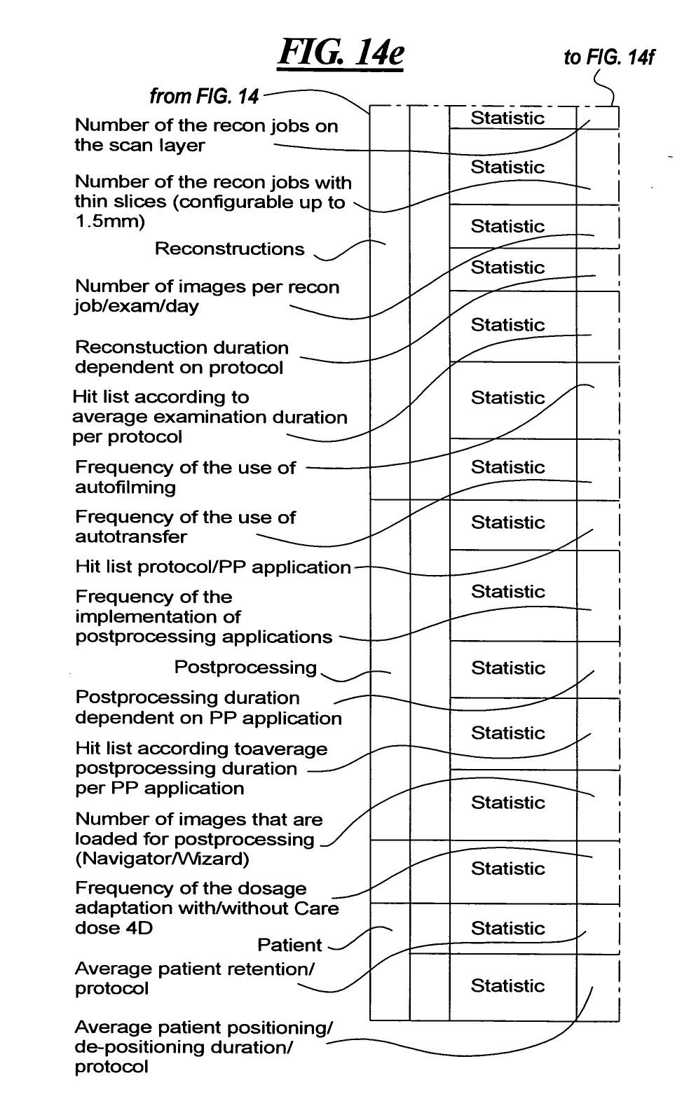

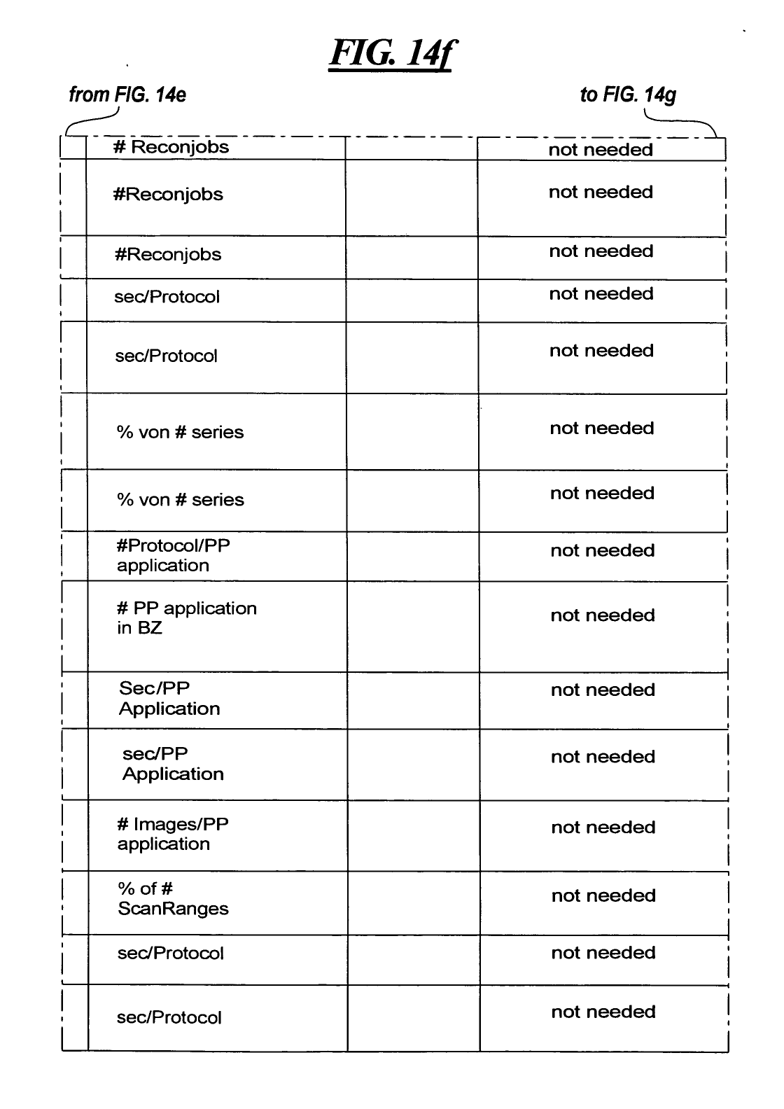

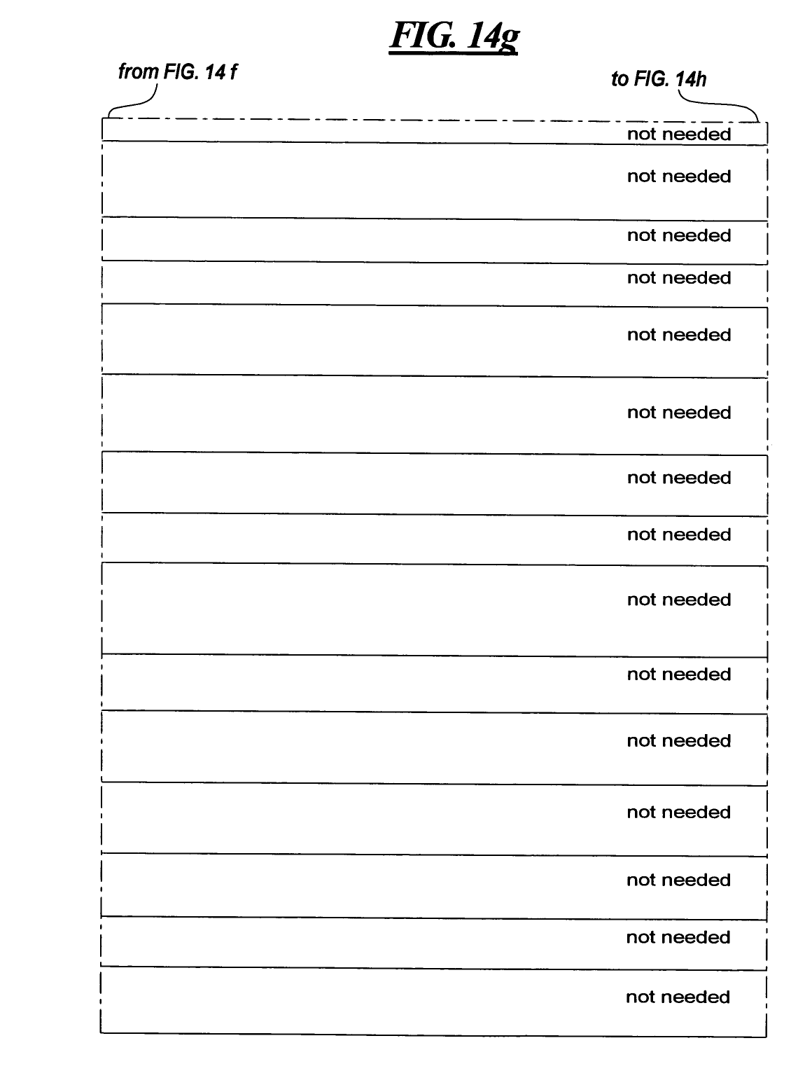

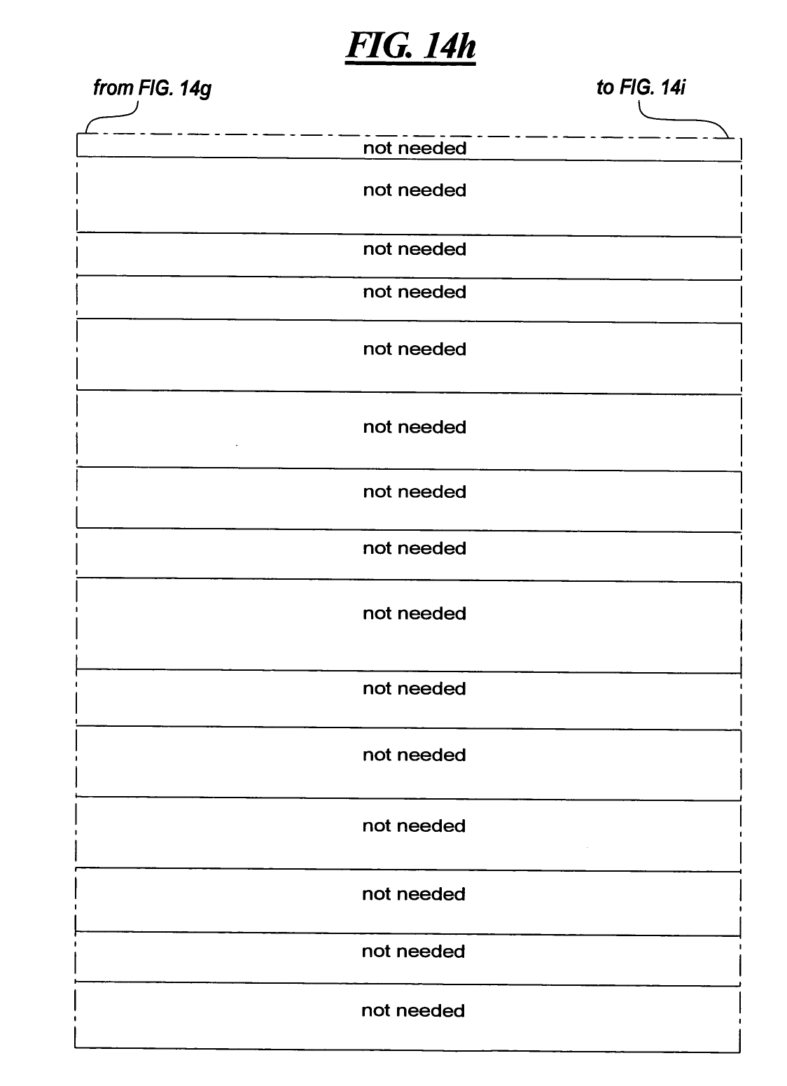

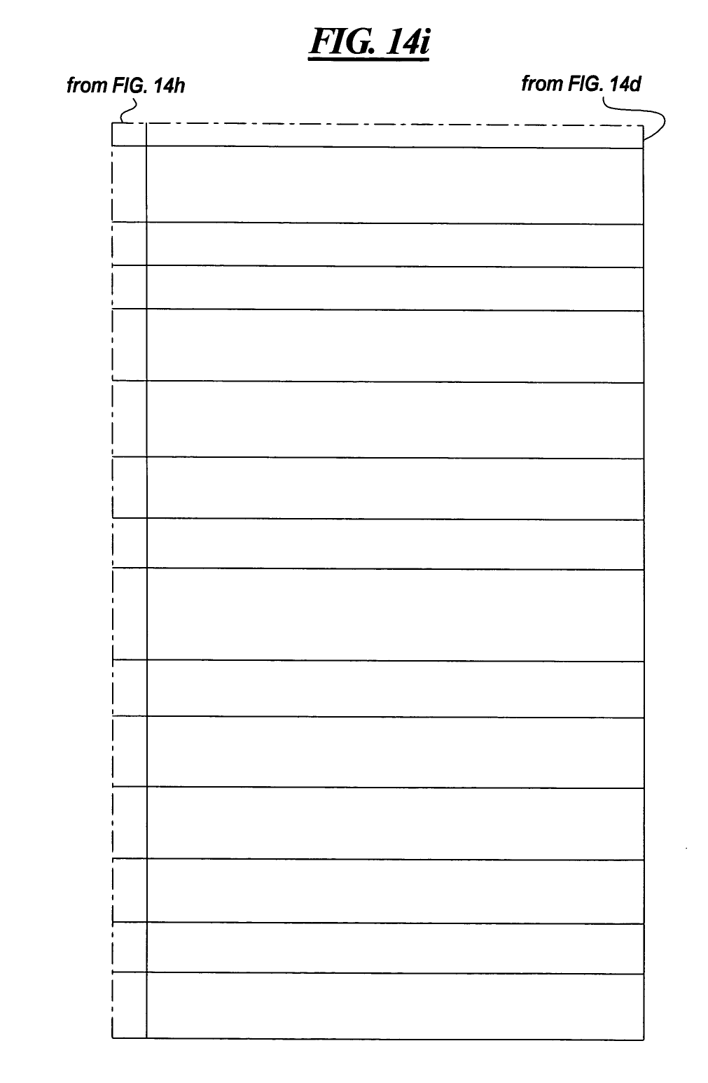

FIGS. 9-14 illustrate examples of workflow reports and parameters according to an example of the present invention.

Although other modifications and changes may be suggested by those skilled in the art, it is the intention of the inventors to embody within the patent warranted hereon all changes and modifications as reasonably and properly come within the scope of their contribution to the art.

We claim:

1. A method for analyzing a workflow of a medical facility relating to technical medical equipment, comprising the steps of:
recording workflow related events relating to use of the technical medical equipment;
storing said workflow related events as data in a file with time stamp information;
performing an analysis of said data to obtain retrospective information on utilization of the technical medical equipment;
comparing said retrospective information on utilization with similar users of the technical medical equipment; and
preparing an output from said comparing.

2. A method as claimed in claim 1, wherein said workflow related events includes demographic data on the medical facility.

3. A method as claimed in claim 1, further comprising the step of: providing a graphical browser displaying the workflow related events.

4. A method as claimed in claim 1, wherein said output is a benchmarking report comparing use of the technical medical equipment of the medical facility to other medical facilities.

5. An apparatus for analysis of workflow through technical medical equipment at a medical facility, comprising:
an automated workflow monitoring system in communication with the technical medical equipment;
a databank receiving workflow data from said automated workflow monitoring system of the medical facility and storing data of other medical facilities;
a benchmarking engine connected to said databank for clustering the medical facility with similar ones of the other medical facilities; and
an output from which is provided a report comparing the workflow of the medical facility to the other medical facilities.

6. A system for display of workflow information for a medical facility, comprising:
a recording of workflow events at the medical facility, said recording includes time stamp information corresponding to the workflow events;
a computer system having a display;
a workflow browser on said workflow system including a graphical representation on said display, said workflow browser displaying workflow events on a time line, said workflow browser displaying events separated into a plurality of event types on said time line, said event types including at least one type selected from the group consisting of: patient based actions, hardware interactions, software interactions, and software functions.

7. A system as claimed in claim 6, wherein further information relating to workflow is displayed along with said time line.

8. A system as claimed in claim 6, wherein said workflow relates to operation of technical medical equipment.

9. A system as claimed in claim 8, wherein said technical medical equipment is a CT scanner and said workflow browser displays events related to use of said CT scanner.

10. A system as claimed in claim 6, wherein said time line is displayed over an arbitrarily defined time interval.

11. A system as claimed in claim 6, wherein said time line is a plurality of time lines corresponding to said plurality of event types, said plurality of time lines being mutually aligned with one another.

12. A system as claimed in claim 6, wherein said workflow browser displays duration of events and pauses between events.

13. A system for display of cumulative workflow information for a medical facility, comprising:
a recording of workflow events at the medical facility, said recording includes time stamp information corresponding to the workflow events;
a computer system having a display;
a cumulative workflow browser on said workflow system including a graphical representation on said display, said cumulative workflow browser displaying combined times of cumulative workflow events over a predetermined time interval, said workflow browser displaying combined times of the events separated into a plurality of event types on said time line, said event types including at least one type selected from the group consisting of: patient based actions, hardware interactions, software interactions, and software functions.

14. A system as claimed in claim 13, wherein said combined times are average times.

15. A system as claimed in claim 13, said workflow events relate to use of technical medical equipment.

16. A system as claimed in claim 15, wherein said technical medical equipment is a CT scanner.

17. A system as claimed in claim 13, wherein said workflow events are viewable by patient examination type.

18. A system as claimed in claim 13, wherein said time line is a plurality of time lines corresponding to said plurality of event types, said plurality of time lines being mutually aligned with one another.

19. A system as claimed in claim 13, wherein said workflow browser displays duration of events and pauses between events.

20. A system for display of workflow information for a medical facility, comprising:
a recording of workflow events at the medical facility utilizing a plurality of technical medical devices, said recording includes time stamp information corresponding to the workflow events;
a computer system having a display;
a workflow browser on said workflow system including a graphical representation on said display, said workflow browser displaying combined workflow events of said plurality of technical medical devices on a time line.

21. A system as claimed in claim 20, wherein said workflow browser displays events separated into a plurality of event types and separated as to each of said plurality of technical medical devices on said time line.

22. A system as claimed in claim 20, wherein said workflow events are separated into different patient procedures, and average times of portions of said different patient procedures are displayed.
